(19) United States
(12) Patent Application Publication
(10) Pub. No.: US 2017/0037072 A1
(43) Pub. Date: Feb. 9, 2017

(54) BIOMASS DIGESTER WITH TWO LIQUID PHASES AND DRAFT TUBE CIRCULATION

(71) Applicant: SHELL OIL COMPANY, Houston, TX (US)

(72) Inventors: Joseph Broun POWELL, Houston, TX (US); Andries Quirin Maria BOON, Houston, TX (US); Ye-Mon CHEN, Sugar Land, TX (US); Glenn Charles KOMPLIN, Katy, TX (US)

(21) Appl. No.: 15/227,990

(22) Filed: Aug. 4, 2016

Related U.S. Application Data
(60) Provisional application No. 62/201,839, filed on Aug. 6, 2015.

(51) Int. Cl. C07H 1/00; B01J 8/24; C10G 3/00; B01J 8/18

(52) U.S. Cl. CPC C07H 1/00; B01J 8/1818; B01J 8/1845; B01J 8/24; C10G 3/49; B01J 2208/00752; B01J 2208/00938; C10G 2400/30; C10G 2400/02; C10G 2400/04; C10G 2300/202

(57) ABSTRACT

A method comprises introducing biomass solids to a digester comprising a reactor, a circulation system including a first injector; providing a catalyst-containing digestion medium and an organic solvent layer floating thereon; circulating the medium through the circulation system; flowing gas through the medium; keeping the medium hot enough to digest the solids; and operating the digester such a headspace exists above the solvent. The digester includes a first eductor having an inlet in the headspace, a second eductor having an inlet in the organic layer, and a downdraft tube having an inlet in the digestion medium. A motive fluid flowing from the first injector draws gas from the headspace into the first eductor, a motive fluid flowing from the first eductor draws fluid from the organic layer into the second eductor, and a motive fluid flowing from the second eductor draws fluid from the digestion medium into the downdraft tube.

### RELATED CASES

The present application claims the benefit of U.S. Provisional Patent Application Ser. No. 62/201,839, filed on Aug. 6, 2015, the entire disclosure of which is hereby incorporated by reference.

### FIELD OF THE INVENTION

The present disclosure generally relates to digestion of cellulosic biomass solids, and, more specifically, to systems and methods in which cellulosic biomass solids may be processed in a hydrothermal digestion unit including one or more internal or external fluid circulation systems.

### BACKGROUND OF THE INVENTION

A number of substances of commercial significance may be produced from natural sources such as biomass. Cellulosic biomass may be particularly advantageous in this regard due to the versatility of the abundant carbohydrates found therein in various forms. As used herein, the term “cellulosic biomass” refers to a living or recently living biological material that contains cellulose. The lignocellulosic material found in the cell walls of higher plants is the world's largest source of carbohydrates. Materials commonly produced from cellulosic biomass may include, for example, paper and pulpwood via partial digestion, and bioethanol by fermentation.

Plant cell walls are divided into two sections: primary cell walls and secondary cell walls. The primary cell wall provides structural support for expanding cells and contains three major polysaccharides (cellulose, pectin, and hemicellulose) and one group of glycoproteins. The secondary cell wall, which is produced after the cell has finished growing, also contains polysaccharides and is strengthened through polymeric lignin that is covalently crosslinked to hemicellulose. Hemicellulose and pectin are typically found in abundance, but cellulose is the predominant polysaccharide and the most abundant source of carbohydrates. The complex mixture of constituents that is co-present with the cellulose can make its processing difficult, as discussed hereinafter.

Significant attention has been placed on developing fossil fuel alternatives derived from renewable resources. Cellulosic biomass has garnered particular attention in this regard due to its abundance and the versatility of the various constituents found therein, particularly cellulose and other carbohydrates. Despite promise and intense interest, the development and implementation of bio-based fuel technology has been slow. Existing technologies have heretofore produced fuels having a low energy density (e.g., bioethanol) and/or that are not fully compatible with existing engine designs and transportation infrastructure (e.g., methanol, biodiesel, hydrogen, and methane). Moreover, conventional bio-based processes have typically produced intermediates in dilute aqueous solutions (>50% water by weight) that are difficult to process further. Energy- and cost-efficient processes for processing cellulosic biomass into fuel blends having similar compositions to fossil fuels would be highly desirable to address the foregoing issues and others.

When converting cellulosic biomass into fuel blends and other materials, cellulose and other complex carbohydrates therein can be extracted and transformed into simpler organic molecules, which can in turn be further reformed thereafter. Fermentation is one process whereby complex carbohydrates from cellulosic biomass may be converted into a more usable form. However, fermentation processes are typically slow, require large volume reactors and high dilution conditions, and produce an initial reaction product having a low energy density (ethanol). Digestion is another way in which cellulose and other complex carbohydrates may be converted into a more usable form. Digestion processes can break down cellulose and other complex carbohydrates within cellulosic biomass into simpler, soluble carbohydrates that are suitable for further transformation through downstream reforming reactions. As used herein, the term “soluble carbohydrates” refers to monosaccharides or polysaccharides that become solubilized in a digestion process. Although the underlying chemistry is understood behind digesting cellulose and other complex carbohydrates and further transforming simple carbohydrates into organic compounds reminiscent of those present in fossil fuels, high-yield and energy-efficient digestion processes suitable for converting cellulosic biomass into fuel blends have yet to be developed. In this regard, the most basic requirement associated with converting cellulosic biomass into fuel blends using digestion and other processes is that the energy input needed to bring about the conversion should not be greater than the available energy output of the product fuel blends. This basic requirement leads to a number of secondary issues that collectively present an immense engineering challenge that has not been solved heretofore.

The issues associated with converting cellulosic biomass into fuel blends in an energy- and cost-efficient manner using digestion are not only complex, but they are entirely different than those that are encountered in the digestion processes commonly used in the paper and pulpwood industry. Since the intent of cellulosic biomass digestion in the paper and pulpwood industry is to retain a solid material (e.g., wood pulp), incomplete digestion is usually performed at relatively low temperatures (e.g., less than about 200° C.) for a fairly short period of time. In contrast, digestion processes suitable for converting cellulosic biomass into fuel blends and other materials are ideally configured to maximize yields by solubilizing as much of the original cellulosic biomass charge as possible in a high-throughput manner. Paper and pulpwood digestion processes also typically remove lignin from the raw cellulosic biomass prior to pulp formation. Although digestion processes used in connection with forming fuel blends and other materials may likewise remove lignin prior to digestion, these extra process steps may impact the energy efficiency and cost of the biomass conversion process. The presence of lignin during high-conversion cellulosic biomass digestion may be particularly problematic in some instances.

Production of soluble carbohydrates for use in fuel blends and other materials via routine modification of paper and pulpwood digestion processes is not believed to be economically feasible for a number of reasons. Simply running the digestion processes of the paper and pulpwood industry for a longer period of time to produce more soluble carbohydrates is undesirable from a throughput standpoint. Use of increased amounts of digestion promoters such as strong alkalis, strong acids, or sulfites to accelerate the digestion rate can increase process costs and complexity due to post-processing separation steps and the possible need to protect downstream components from these agents. Accelerating the digestion rate by increasing the digestion temperature can actually reduce yields due to thermal degradation of soluble carbohydrates that can occur at elevated digestion temperatures, particularly over extended periods of time. Once produced by digestion, soluble carbohydrates are very reactive and can rapidly degrade to produce caramelans and other heavy ends degradation products, especially under higher temperature conditions, such as above about 150° C. Any of these difficulties can impede the economic viability of fuel blends derived from cellulosic biomass.

One way in which soluble carbohydrates can be protected from thermal degradation is through subjecting them to one or more catalytic reduction reactions, which may include hydrogenation and/or hydrogenolysis reactions. Stabilizing soluble carbohydrates through conducting one or more catalytic reduction reactions may allow digestion of cellulosic biomass to take place at higher temperatures than would otherwise be possible without unduly sacrificing yields. Depending on the reaction conditions and catalyst used, reaction products formed as a result of conducting one or more catalytic reduction reactions on soluble carbohydrates may comprise one or more alcohol functional groups, particularly including triols, diols, monohydric alcohols, and any combination thereof, some of which may also include a residual carbonyl functionality (e.g., an aldehyde or a ketone). Such reaction products are more thermally stable than soluble carbohydrates and may be readily transformable into fuel blends and other materials through conducting one or more downstream reforming reactions. In addition, the foregoing types of reaction products are good solvents in which a hydrothermal digestion may be performed, thereby promoting solubilization of soluble carbohydrates as their reaction products during hydrothermal digestion.

A particularly effective manner in which soluble carbohydrates may be formed and converted into more stable compounds is through conducting the hydrothermal digestion of cellulosic biomass in the presence of molecular hydrogen and a slurry catalyst capable of activating the molecular hydrogen (also referred to herein as a “hydrogen-activating catalyst”). That is, in such approaches (termed “in situ catalytic reduction reaction processes” herein), the hydrothermal digestion of cellulosic biomass and the catalytic reduction of soluble carbohydrates produced therefrom may take place in the same vessel. As used herein, the term “slurry catalyst” will refer to a catalyst comprising fluidly mobile catalyst particles that can be at least partially suspended in a fluid phase via gas flow, liquid flow, mechanical agitation, or any combination thereof. If the slurry catalyst is sufficiently well distributed in the cellulosic biomass, soluble carbohydrates formed during hydrothermal digestion may be intercepted and converted into more stable compounds before they have had an opportunity to significantly degrade, even under thermal conditions that otherwise promote their degradation. Without adequate catalyst distribution, soluble carbohydrates produced by in situ catalytic reduction reaction processes may still degrade before they have had an opportunity to encounter a catalytic site and undergo a stabilizing reaction. In situ catalytic reduction reaction processes may also be particularly advantageous from an energy efficiency standpoint, since hydrothermal digestion of cellulosic biomass is an endothermic process, whereas catalytic reduction reactions are exothermic. Thus, the excess heat generated by the in situ catalytic reduction reaction(s) may be utilized to drive the hydrothermal digestion with little opportunity for heat transfer loss to occur, thereby lowering the amount of additional heat energy input needed to conduct the digestion.

Another issue associated with the processing of cellulosic biomass into fuel blends and other materials is created by the need for high conversion percentages of a cellulosic biomass charge into soluble carbohydrates. Specifically, as cellulosic biomass solids are digested, their size gradually decreases to the point that they can become fluidly mobile. As used herein, cellulosic biomass solids that are fluidly mobile, particularly cellulosic biomass solids that are about 3 mm in size or less, will be referred to as “cellulosic biomass fines.” Cellulosic biomass fines can be transported out of a digestion zone of a system for converting cellulosic biomass and into one or more zones where solids are unwanted and can be detrimental. For example, cellulosic biomass fines have the potential to plug catalyst beds, transfer lines, valving, and the like. Furthermore, although small in size, cellulosic biomass fines may represent a non-trivial fraction of the cellulosic biomass charge, and if they are not further converted into soluble carbohydrates, the ability to attain a satisfactory conversion percentage may be impacted. Since the digestion processes of the paper and pulpwood industry are run at relatively low cellulosic biomass conversion percentages, smaller amounts of cellulosic biomass fines are believed to be generated and have a lesser impact on those digestion processes.

In addition to the desired carbohydrates, other substances may be present within cellulosic biomass that can be especially problematic to deal with in an energy- and cost-efficient manner. Sulfur- and/or nitrogen-containing amino acids or other catalyst poisons may be present in cellulosic biomass. If not removed, these catalyst poisons can impact the catalytic reduction reaction(s) used to stabilize soluble carbohydrates, thereby resulting in process downtime for catalyst regeneration and/or replacement and reducing the overall energy efficiency when restarting the process. This issue is particularly significant for in situ catalytic reduction reaction processes, where there is minimal opportunity to address the presence of catalyst poisons, at least without significantly increasing process complexity and cost, but can be mitigated through various means, such as catalyst selection. For example, a poison-tolerant or high-activity catalyst can provide effective conversions, even in the presence of lignin. Detailed discussion of catalyst selection is disclosed elsewhere and is beyond the scope of this specification. Also, as mentioned above, lignin can also be particularly problematic to deal with if it is not removed prior to beginning digestion. During cellulosic biomass processing, the significant quantities of lignin present in cellulosic biomass may lead to fouling of processing equipment, potentially leading to costly system down time. Significant lignin quantities can also lead to realization of a relatively low conversion of the cellulosic biomass into useable substances per unit weight of feedstock. The effects of lignin can be mitigated through use of one or more lignin solvents. Lignin mitigation is disclosed elsewhere and is beyond the scope of this specification

Further information relating to the present technology can be found in commonly-owned U.S. application Ser. No. 14/264,647, which is incorporated herein by reference in its entirety.

As evidenced by the foregoing, the efficient conversion of cellulosic biomass into fuel blends and other materials is a complex problem that presents immense engineering challenges. The present disclosure addresses these challenges and provides related advantages as well.

### SUMMARY OF THE INVENTION

The present disclosure generally relates to digestion of cellulosic biomass solids, and, more specifically, to systems and methods in which cellulosic biomass solids may be processed in a hydrothermal digestion unit having a reactor packing material present therein.

In some embodiments, the present invention provides methods comprising: a) introducing cellulosic biomass solids to a hydrothermal digestion unit comprising i) a reactor, ii) a gas feed line for providing gas to the reactor, iii) a biomass feed system for feeding biomass into the reactor, iv) a fluid circulation system including a fluid inlet, a pump, and a first fluid injector, wherein at least the fluid inlet and the first fluid injector are in fluid communication with the pump and are within the reactor; and v) a screen positioned within the reactor and defining a lower zone therebelow; b) providing a liquid phase digestion medium containing a slurry catalyst in the hydrothermal digestion unit, the slurry catalyst being capable of activating molecular hydrogen; c) providing a layer of organic solvent in the hydrothermal digestion unit, the organic solvent being less dense than and substantially immiscible with the digestion medium, whereby the organic solvent forms an organic layer floating on the digestion medium; d) circulating the liquid phase digestion medium through the fluid circulation system; e) supplying an upwardly directed flow of molecular hydrogen through the cellulosic biomass solids; f) maintaining the cellulosic biomass solids and slurry catalyst at a temperature sufficient to cause digestion of cellulosic biomass solids into an alcoholic component; and g) operating the hydrothermal digestion unit such that the level of the organic layer in the reactor is below the top of the reactor, thereby leaving a gas-filled headspace; h) wherein the hydrothermal digestion unit further comprises 1) at least one first eductor having a first inlet in the headspace and an outlet below the first inlet, 2) at least one second eductor having a second inlet in the organic layer and an outlet below the second inlet end, 3) at least one downdraft tube having a third inlet in the digestion medium and an outlet in the lower zone, wherein fluid flowing from the first fluid injector draws gas from the headspace into the first eductor, fluid flowing out of the first eductor draws fluid from the organic layer into the second eductor, fluid flowing out of the second eductor draws fluid from the digestion medium into the downdraft tube, and fluid flowing out of the downdraft tube is discharged into the reactor below the packing material.

The fluid circulation system may removes slurry from the lower zone via the fluid inlet and may transmit the removed slurry to a separator that separates the slurry into a catalyst concentrate stream and a stream that is relatively low in solids. The catalyst concentrate stream may be fed back into the reactor at a point that is above the packed bed and the low-solids stream may be injected into the reactor via the first fluid injector. The method may further include the step of operating the system such that the flow rate of fluids in the draft tube is sufficient to carry the entrained gas to the lower zone.

At least a portion of either the fluid circulation system or the downdraft tube is outside of the reactor. From one-half up to all of the volume of slurry removed from the lower zone is returned to reactor via the first fluid injector. The method may further include the step of removing a portion of the organic layer from the reactor, removing lignin therefrom so as to generate a lignin-reduced organic steam, and returning at least a portion of the lignin-reduced organic stream to the reactor.

The features and advantages of the present disclosure will be readily apparent to one having ordinary skill in the art upon a reading of the description of the embodiments that follows.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and the benefit of this disclosure.

FIG. 1 is a schematic illustration of a hydrothermal digestion unit including a reactor packing material and an updraft tube;

FIG. is a schematic illustration of a stinger unit in accordance with one embodiment of the present invention;

FIG. 3 is a schematic illustration of a hydrothermal digestion unit including a reactor packing material, an updraft tube, and a downdraft tube;

FIG. 4 is a schematic illustration of a hydrothermal digestion unit including a reactor packing material, a downdraft tube, and a pair of eductors for entraining gas and liquid into the downdraft tube; and

FIG. 5 is a schematic illustration of a hydrothermal digestion unit including a reactor packing material, a downdraft tube, and a three eductors for entraining gas and liquid phases in the downdraft tube.

### DETAILED DESCRIPTION

The present disclosure generally relates to digestion of cellulosic biomass solids, and, more specifically, to systems and methods in which cellulosic biomass solids may be processed in a hydrothermal digestion unit having a reactor packing material present therein.

In the embodiments described herein, the digestion rate of cellulosic biomass solids may be accelerated in the presence of a liquid phase digestion medium comprising a digestion solvent. In some instances, the liquid phase digestion medium may be maintained at elevated pressures that keep the digestion solvent in a liquid state when raised above its normal boiling point. Although the more rapid digestion rate of cellulosic biomass solids under elevated temperature and pressure conditions may be desirable from a throughput standpoint, soluble carbohydrates may be susceptible to degradation at elevated temperatures, as discussed above. As further discussed above, one approach for addressing the degradation of soluble carbohydrates during hydrothermal digestion is to conduct an in situ catalytic reduction reaction process so as to convert the soluble carbohydrates into more stable compounds as soon as possible after their formation.

Although digesting cellulosic biomass solids by an in situ catalytic reduction reaction process may be particularly advantageous for at least the reasons noted above, successfully executing such a coupled approach may be problematic in other aspects. One significant issue that may be encountered is that of adequate catalyst distribution within the digesting cellulosic biomass solids, since insufficient catalyst distribution can result in poor stabilization of soluble carbohydrates. The present inventors discovered that, in certain instances, a slurry catalyst may be effectively distributed from the bottom of a charge of cellulosic biomass solids to the top using upwardly directed fluid flow to fluidize and upwardly convey slurry catalyst particulates into the interstitial spaces within the charge. Suitable techniques for using fluid flow to distribute a slurry catalyst within cellulosic biomass solids in such a manner are described in commonly-owned U.S. application Ser. Nos. 13/928,877 and 13/928,770, which are each filed on Jun. 27, 2013 and incorporated herein by reference in their entireties. In addition to affecting distribution of the slurry catalyst, upwardly directed fluid flow may promote expansion of the cellulosic biomass solids and disfavor gravity-induced compaction that occurs during their addition and digestion, particularly as the digestion process proceeds and their structural integrity decreases. Such approaches may also address the problem of cellulosic biomass fines, since they may be co-flowed with the motive fluid.

Effective distribution of molecular hydrogen within cellulosic biomass solids during hydrothermal digestion can also be problematic, as described in commonly owned U.S. application Ser. Nos. 14/108,968 and 14/108,933, each filed on filed on Dec. 17, 2013 and incorporated herein by reference in its entirety. As with a poorly distributed slurry catalyst, inadequate distribution of molecular hydrogen in cellulosic biomass solids can likewise result in poor stabilization of soluble carbohydrates during in situ catalytic reduction reaction processes. Without being bound by any theory or mechanism, it is believed that a poor distribution of molecular hydrogen within cellulosic biomass solids may be realized due to a coalescence of introduced molecular hydrogen into large bubbles that are unable to penetrate into the interstitial spaces within a charge of digesting cellulosic biomass solids. As the vertical height of a charge of cellulosic biomass solids in contact with a continuous liquid phase increases, the propensity toward hydrogen bubble coalescence may be increased.

The present inventors recognized that the problems of biomass compaction and molecular hydrogen distribution might be simultaneously addressed by altering the configuration of a hydrothermal digestion unit used to digest cellulosic biomass solids to include a charge of reactor packing material therein. In some instances, such a configuration will be referred to herein as a “packed digester.” By digesting a charge of cellulosic biomass solids in a packed digester, the flow of the biomass particles through the digester is altered and, preferably, slowed as compared to flow through an equivalent non-packed configuration. As the cellulosic biomass solids are denser than the digestion medium, they tend to drift downward through the unit and, without the packing material to provide partial support, would settle in a compacting mass. Thus, for a fixed vertical height, a packed digester may provide improved contact between biomass, catalyst, hydrogen, and liquid solvent in the reactor than a non-packed digestion unit.

In addition, use of a packed reactor may reduce the likelihood of hydrogen bubble coalescence. More particularly, hydrogen bubbles that coalesce as they flow upward from a source disposed at the bottom of a packed digester may be re-distributed in the cellulosic biomass solids as they pass through the reactor packing. Furthermore, when molecular hydrogen is introduced to a packed digester, the upflow of hydrogen gas may be more likely to maintain an effective slurry catalyst distribution than would be possible when fluidizing the slurry catalyst through a mass of settled cellulosic biomass solids, such as would typically accumulate in a non-packed hydrothermal digestion unit.

In addition to better promoting the distribution of a slurry catalyst and molecular hydrogen in the cellulosic biomass solids during hydrothermal digestion, a packed digester may also better address the problem of biomass compaction. In a non-packed vertical hydrothermal digester, as the vertical height of a charge of cellulosic biomass solids increases, the lower portions of the charge can become compacted by the weight of the upper portions of the charge. This problem can be particularly significant as the hydrothermal digestion process progresses and the structural integrity of the cellulosic biomass solids decreases, leading to formation of a mush-like state, in which it is difficult to distribute a slurry catalyst and molecular hydrogen due to a reduced access to interstitial spaces therein. In contrast, by conducting the hydrothermal digestion of cellulosic biomass solids in a packed digester, compaction forces on the lower portions of the cellulosic biomass solids may be conferred to the packing material in the hydrothermal digestion unit, thereby lowering the likelihood of excessive compaction.

In some embodiments, the reactor packing material and/or at least a portion of the cellulosic biomass solids may reside on a porous retention structure or screen that is configured to allow the upwardly directed flow of molecular hydrogen to pass therethrough. Suitable porous retention structures can include, for example, screens, grids, and like porous media. In various embodiments, the porous retention structure may reside within the continuous liquid phase. As cellulosic biomass solids are at least partially digested, they may lose structural integrity and attain a mush-like consistency that can block fluid flow pathways within the remainder of the cellulosic biomass solids. However, by including a packing material in the digester, the biomass solids will be retained in the packed zone while they are digested. After sufficient digestion, at least a portion of the cellulosic biomass solids may pass through the packing material and the retention structure and enter the space below the porous retention structure. Passage of the partially digested cellulosic biomass solids through the porous retention structure may be aided by the circulating flow of gas and/or liquid within the digester. By keeping the porous retention structure free of smaller particles, there may be a reduced likelihood of undesirably restricting flow in the hydrothermal digestion unit. In the foregoing concept, sometimes referred to as an “open screen” approach, cellulosic biomass solids collect on the porous retention structure in a sufficient quantity to form a filter cake that promotes retention of the remaining cellulosic biomass solids, regardless of particle size, until the filter cake particles are reduced in size and fall through and/or are extruded through the pores of the porous retention structure.

In addition to the foregoing advantages, an packed digester may remain compatible with techniques used for addressing the formation of heterogeneous liquid phases during hydrothermal digestion of cellulosic biomass solids. While digesting cellulosic biomass solids by an in situ catalytic reduction reaction process in the presence of a slurry catalyst and an aqueous phase digestion solvent, where the cellulosic biomass solids were supplied on an ongoing basis, the present inventors discovered that lignin from the cellulosic biomass solids eventually separated as a phenolics liquid phase that was neither fully dissolved nor fully precipitated, but instead formed as a discrete liquid phase that was highly viscous and hydrophobic. The slurry catalyst was well wetted by the phenolics liquid phase and accumulated therein over time, thereby making the slurry catalyst less readily distributable in the cellulosic biomass solids (e.g., by using upwardly directed fluid flow). In many instances, the phenolics liquid phase was located below the aqueous phase, which also contained an alcoholic component derived from the cellulosic biomass solids via a catalytic reduction reaction of soluble carbohydrates.

Depending on the ratio of water and organic solvent in the digestion solvent, rates of fluid flow, catalyst identity, reaction times and temperatures, and the like, a light organics phase was also sometimes observed, typically located above the aqueous phase, where the components of the light organics phase were also derived, at least in part, from the cellulosic materials in the biomass. Components present in the light organics phase included, for example, the alcoholic component derived from the cellulosic biomass solids, including C₄ or greater alcohols, and self-condensation products, such as those obtained by the acid-catalyzed Aldol reaction. The alcoholic component in the resulting two- or three-phase liquid mixture may be processed as described in more detail in commonly owned U.S. application Ser. Nos. 14/067,501 and 14/067,330, each filed on Oct. 30, 2013 and incorporated herein by reference in its entirety.

Techniques for mitigating the accumulation of a slurry catalyst in a phenolics liquid phase are described in more detail in commonly owned U.S. patent application Ser. No. 14/067,309, filed on Oct. 30, 2013 and incorporated herein by reference in its entirety. As described therein, the accumulated slurry catalyst within the phenolics liquid phase may be conveyed from a lower portion of the hydrothermal digestion unit to a location above the cellulosic biomass solids and released, such that the slurry catalyst then contacts the cellulosic biomass solids. By conveying the accumulated slurry catalyst in such a manner, the slurry catalyst may become redistributed in the cellulosic biomass solids as the phenolics liquid phase percolates downward through the cellulosic biomass solids, rather than from becoming distributed via upwardly directed fluid flow. As described herein, such techniques may be practiced in a similar manner when hydrothermal digestion is performed using a packed digester.

Unless otherwise specified, it is to be understood that use of the terms “biomass” or “cellulosic biomass” in the description herein refers to “cellulosic biomass solids.” Solids may be in any size, shape, or form. The cellulosic biomass solids may be natively present in any of these solid sizes, shapes, or forms, or they may be processed prior to hydrothermal digestion. In some embodiments, the cellulosic biomass solids may be chopped, ground, shredded, pulverized, and the like to produce a desired size prior to hydrothermal digestion. In the text below, the size of the biomass particles may be described in terms of their average greatest dimension, which refers to the average over multiple particles of the longest dimension of each particle. In some or other embodiments, the cellulosic biomass solids may be washed (e.g., with water, an acid, a base, combinations thereof, and the like) prior to hydrothermal digestion taking place.

In practicing the present embodiments, any type of suitable cellulosic biomass source may be used. Suitable cellulosic biomass sources may include, for example, forestry residues, agricultural residues, herbaceous material, municipal solid wastes, waste and recycled paper, pulp and paper mill residues, and any combination thereof. Thus, in some embodiments, a suitable cellulosic biomass may include, for example, corn stover, straw, bagasse, miscanthus, sorghum residue, switch grass, bamboo, water hyacinth, hardwood, hardwood chips, hardwood pulp, softwood, softwood chips, softwood pulp, and any combination thereof. Leaves, roots, seeds, stalks, husks, and the like may be used as a source of the cellulosic biomass. Common sources of cellulosic biomass may include, for example, agricultural wastes (e.g., corn stalks, straw, seed hulls, sugarcane leavings, nut shells, and the like), wood materials (e.g., wood or bark, sawdust, timber slash, mill scrap, and the like), municipal waste (e.g., waste paper, yard clippings or debris, and the like), and energy crops (e.g., poplars, willows, switch grass, alfalfa, prairie bluestream, corn, soybeans, and the like). The cellulosic biomass may be chosen based upon considerations such as, for example, cellulose and/or hemicellulose content, lignin content, growing time/season, growing location/transportation cost, growing costs, harvesting costs, and the like.

Illustrative carbohydrates that may be present in cellulosic biomass solids include, for example, sugars, sugar alcohols, celluloses, lignocelluloses, hemicelluloses, and any combination thereof. Once soluble carbohydrates have been produced through hydrothermal digestion according to the embodiments described herein, the soluble carbohydrates may be transformed into a more stable reaction product comprising a monohydric alcohol, a glycol, a triol, or any combination thereof, at least some of which may also contain a carbonyl functionality. As used herein, the term “glycol” will refer to compounds containing two alcohol functional groups, two alcohol functional groups and a carbonyl functionality, or any combination thereof. As used herein, the term “carbonyl functionality” will refer to an aldehyde functionality or a ketone functionality. As used herein, the term “triol” will refer to compounds containing three alcohol functional groups, three alcohol functional groups and a carbonyl functionality, and any combination thereof. As used herein, the term “monohydric alcohol” will refer to compounds containing one alcohol functional group, one alcohol functional group and a carbonyl functionality, and any combination thereof.

As used herein, the term “phenolics liquid phase” will refer to a fluid phase comprising liquefied lignin. In some embodiments, the phenolics liquid phase may be more dense than water, but it may also be less dense than water depending on lignin concentrations and the presence of other components, for example.

As used herein, the term “alcoholic component” will refer to a monohydric alcohol, glycol, triol, or any combination thereof that is formed from a catalytic reduction reaction of soluble carbohydrates derived from cellulosic biomass solids.

As used herein, the term “light organics phase” will refer to a fluid phase that is typically less dense than water and comprises an organic compound. The organic compound may include at least a portion of the alcoholic component formed via catalytic reduction of soluble carbohydrates, which may include C₄ or greater alcohols and self-condensation products thereof.

As used herein, the term “vertical” will refer to a surface or structure oriented at an angle of between about 85 degrees and about 90 degrees relative to horizontal.

As used herein, the term “tubular” will refer to an elongated three-dimensional structure having an open space therein. Any number of surfaces may be present within the open space within the interior of the tubular structure. That is, the term “tubular” may be used to refer to both cylindrical and prismatic elongated three-dimensional structures. In embodiments where a tubular structure is cylindrical, it may have a length that is greater than its diameter.

As used herein, the term “upwardly directed” will refer to a direction of fluid flow that is opposite to the direction of the gravitational force.

In some embodiments, methods described herein can comprise: introducing cellulosic biomass solids to a packed digester; introducing a liquid phase digestion medium containing a slurry catalyst to the digester, the slurry catalyst being capable of activating molecular hydrogen; wherein, once introduced to the hydrothermal digestion unit, the cellulosic biomass solids, the liquid phase digestion medium, and the slurry catalyst flow through the digester; supplying an upwardly directed flow of molecular hydrogen through the cellulosic biomass solids as they descend through the digester; and heating the cellulosic biomass solids as they descend through the reactor packing material in the presence of the slurry catalyst and the molecular hydrogen, thereby forming an alcoholic component derived from the cellulosic biomass solids.

In some embodiments, the upwardly directed flow of molecular hydrogen through the cellulosic biomass solids may be supplied from a gas distribution system that is disposed at the bottom of the digester, or at multiple locations within the reactor. As described above, molecular hydrogen so introduced may mediate stabilization of soluble carbohydrates both by serving as a reactant for a catalytic reduction reaction and promoting distribution of a slurry catalyst in the cellulosic biomass solids. Suitable gas distribution systems may include slotted distributors, manifolds, empty piping with an array of holes disposed thereon, sintered metal elements, collections of nozzles at a spacing effective to disperse a gas phase, other gas distribution manifolds, combinations thereof, and the like.

In some embodiments, molecular hydrogen being supplied to the gas distribution system may be supplied from a molecular hydrogen source external to the hydrothermal digestion unit. In some or other embodiments, the molecular hydrogen being supplied to the gas distribution system may be recirculated or recycled from one section of the hydrothermal digestion unit to another.

### Digester

Various exemplary embodiments of the biomass conversion systems will now be further described with reference to the drawings. When like elements are used in one or more figures, identical reference characters will be used in each figure, and a detailed description of the element will be provided only at its first occurrence. Some features of the biomass conversion systems may be omitted in certain depicted configurations in the interest of clarity. Moreover, certain features such as, but not limited to pumps, valves, gas bleeds, gas inlets, fluid inlets, fluid outlets and the like have not necessarily been depicted in the figures, but their presence and function will be understood by one having ordinary skill in the art. In the figures, arrows have been drawn to depict the direction of liquid or gas flow.

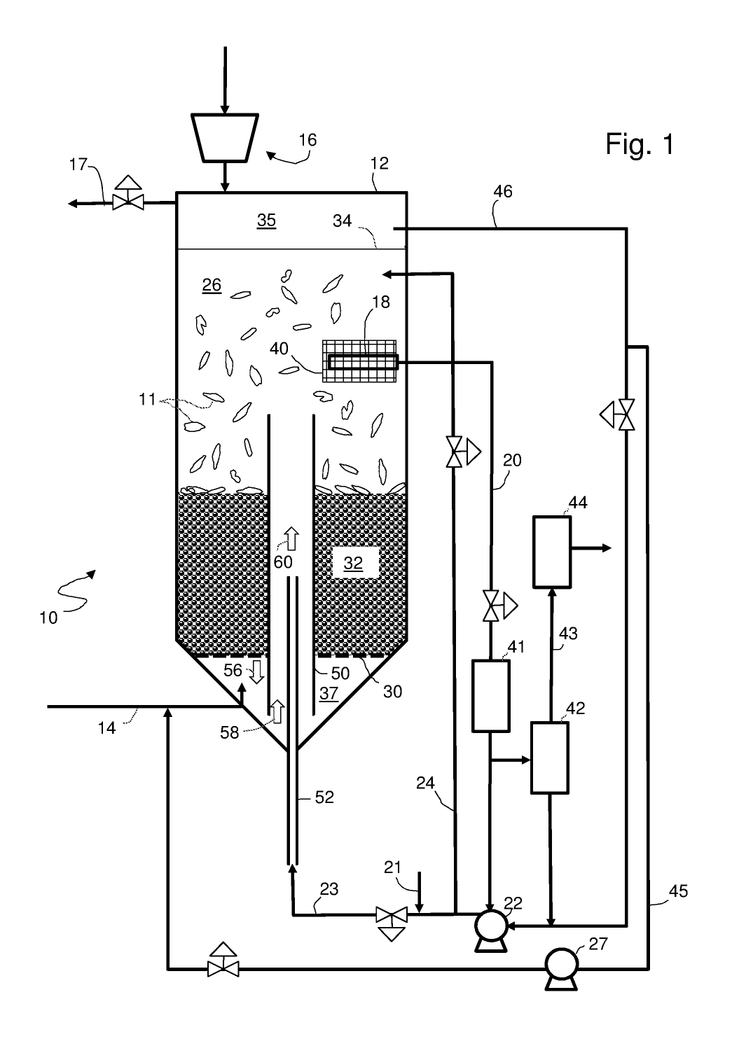

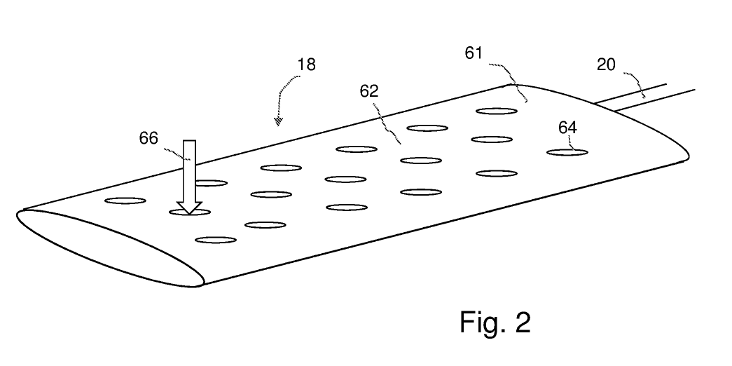

Referring initially to FIG. 1, an exemplary biomass conversion system 10 in which cellulosic biomass solids 11 may be digested in the presence of a liquid phase and a gas phase that are interfacial to one another comprises a hydrothermal digestion unit or reactor 12 into which a biomass introduction mechanism 16 and a gas feed line 14 discharge. If desired, system 10 may also include a gas vent line 17 in communication with the top of reactor 12, through which gas may be vented. If desired, gas feed line 14 may feed gas into reactor 12 via a sparger or other distribution means (not shown), such as are known. Likewise, solids introduction mechanism 16 may include means (not shown) for raising the pressure of the cellulosic biomass solids from atmospheric pressure to a pressure near that of the operating pressure of hydrothermal digestion unit 12, thereby allowing continuous or semi-continuous introduction of cellulosic biomass solids to take place without fully depressurizing hydrothermal digestion unit 12. Suitable loading mechanisms and pressure transition zones are known.

In some preferred embodiments, hydrothermal digestion unit 12 contains a retention structure or screen 30, a mass of packing material 32 resting on screen 30, a lower zone 37 below screen 30, a draft tube 50 extending through screen 30 and packing material 32, a liquid intake 18, an optional headspace 35, and a catalyst-containing liquid slurry 26 having an upper surface 34. In preferred embodiments, the liquid level is maintained such that liquid surface 34 is above the top of the mass of packing material 32 and packing material 32 is completely immersed in the liquid.

Liquid intake 18 is preferably positioned so as to be below the liquid surface 34 and above packing material 32 and is fluidly connected to a flow line 20, which is in turn connected to a pump 22. Liquid intake 18 is preferably provided with a surrounding cage 40, which prevents solids in the slurry 26 from clogging intake 18. Optionally, an additional outflow line 46 may be in fluid communication with the inside volume of the digestion unit 12 at a point that is preferably above the upper intake 18 and, if optional headspace 35 is present, in fluid communication with headspace 35. Flow line 46 may be used to recirculate gas from headspace 35 back into feed gas line 14, via line 45 and compressor 27, which is preferably a blower but may be provided with an optional cooler and condenser (not shown). Additionally or alternatively, flow line 46 may feed into pump 22. The pressurized output of pump 22 flows via line 23 into lower zone 37 of digestion unit 12 as described in greater detail below. In preferred embodiments, a recycle line 24 comes off of line 22 and discharges into the top half of reactor 12 above the packing material 32.

If desired, at least a second bed of packing material (not shown) can be included in the reactor. Liquid intake 18 may be positioned above the top bed or between the beds. Draft tube 50 may likewise extend from lower zone 37 through one or both beds.

The nature of packing material 32 is not particularly limited. In some embodiments, the packing material comprises a known reactor packing material such as rings, saddles, spirals, doughnuts, or other geometrically regular and/or irregular forms, and may for example comprise Pall rings, Raschig rings, structured packing, or any other commercially available packing material. Packing material 32 preferably has a high surface to volume ratio and is sized so that the it has an average greatest dimension that is between 20% and 500% of the average greatest dimension of the cellulosic biomass solids entering the digester, so that it can more effectively prevent compaction of the biomass solids while also allowing circulation of the slurry catalyst and disrupting the coalescence of gas bubbles. In other embodiments, reactor packing material 32 may comprise comprises structured packing. The packing material may be placed or dumped into the reactor. The portion of the reactor that is occupied by the packing material may be referred to as the “packed zone.” In some or other embodiments, the height of the packed zone is at least 25% of the height of the reactor and may be at least 90% of the height of the reactor. The reactor packing material 32 preferably has a skeletal density at least as great as the density of the liquid digestion medium.

As mentioned above, the liquid slurry 26 may have a slurry catalyst distributed therein while in hydrothermal digestion unit 12. In the interest of clarity, particulates of the slurry catalyst have not been depicted in the Figures.

Once introduced to hydrothermal digestion unit 12, cellulosic biomass solids 11 drift downward through the slurry 26 and may, depending on flow conditions, come to rest on screen 30 and/or on the reactor packing material. Weakened and/or partially digested cellulosic biomass solids may pass through screen 30 and enter lower zone 37. The weakened or partially digested cellulosic biomass solids may be strands or fibers, or may break apart into finely divided particulates as they pass through screen 30.

An upwardly directed flow of molecular hydrogen may be supplied to hydrothermal digestion unit 12 via feed line 14. Feed line 14 may be connected to a flow dispersal system (not shown) within hydrothermal digestion unit 12 that results in formation of hydrogen gas bubbles within the liquid phase. As the bubbles rise within reactor 12 they contribute to turbulence within the liquid phase and help disperse the biomass solids in the liquid phase. The bubbles may also coalesce. The bubbles ultimately exit the liquid phase to form a gas phase in optional headspace 35.

In addition to the upwardly directed flow of molecular hydrogen, turbulence in the liquid phase can be increased by means of an upwardly directed liquid stream supplied to hydrothermal digestion unit 12 by line 23. Fluid in line 23 preferably comes from recycle line 20 via pump 22 and, optionally, gas from line 46.

In some embodiments, the fluid circulation system may also include a recycle fluid outlet positioned in the reactor. This may be, for example a recycle line 24 in communication with line 23 downstream of pump 22. The outlet of recycle line 24 may be above the packed zone 32.

### Stinger

Referring briefly to FIG. 2, in some embodiments liquid intake 18 includes an inlet or stinger unit 61 having an upper surface 62, an inner volume in communication with said catalyst circulation system via line 20, and at least one opening 64 in upper surface 62 for allowing fluid to enter said inner volume. Depending on the tolerance of pump 22, stinger unit 61 may optionally be enclosed in a cage filter (shown schematically at 40 in FIG. 1) that provides a filtered volume adjacent to opening(s) 64, thereby reducing the likelihood that opening(s) 64 will become clogged with partially digested biomass solids. In operation, the liquid slurry comprising liquid phase digestion medium and catalyst particles flow through said cage filter and then into the catalyst circulation system via opening(s) 64 in catalyst stinger unit 61, as shown at arrow 66.

Stinger unit 61 may have any number of openings and preferably has at least two openings, more preferably at least six openings, and still more preferably at least 24 openings. Depending on the equipment to be protected, each opening 64 may have a largest dimension of no greater than the tolerance of the pump. For example, openings may be smaller than 10 cm, optionally smaller than 5 cm, optionally smaller than 2 cm, or in some instances smaller than 0.5 cm. Opening(s) 64 are preferably only on the upper surface of stinger 61, so as to avoid the entry of gas bubbles into the liquid circulation system. Additionally or alternatively, stinger 61 may be positioned in an alcove or dead space within digester 12, which may in turn provide some protection from the ingress of biomass solids by a screen.

If a cage filter 40 is present, the volume enclosed by filter 40 is at preferably least 50% greater than the inner volume of stinger unit 61 and more preferably at preferably least twice the inner volume of stinger unit 61. In some embodiments, cage filter 40 may include a plurality of openings each having a largest dimension no greater than 1.0 cm and more preferably less than 0.5 cm. It may be desirable to reverse the flow of slurry through the circulation system from time to time so as to remove accumulated solids from stinger 61, in which case slurry would flow out of opening(s) 64. In some preferred embodiments, stinger 18 is in an alcove (nor shown) or otherwise separated from main body of the liquid slurry so as to reduce its contact with biomass solids and gas bubbles.

### Updraft System

Referring again to FIG. 1, in preferred embodiments, a portion of the liquid phase removed from digester 12 via intake 18 and conveyed by line 20 to pump 22 can be pumped at high pressure back into digester 12 via line 23. Line 23 preferably discharges fluid into digester 12 via an injector means 52, which may be a nozzle, a tube, or merely an extension of line 22. The liquid supplied by line 23 may receive make-up liquid via a line 21 so as to replace the portion of the liquid phase that is removed for downstream processing, as discussed in more detail hereinafter.

In preferred embodiments, the lower end of draft tube 50 is positioned in lower zone 37 and the upper end of draft tube 50 is above the mass of reactor packing material but below the liquid surface 34. Draft tube 50 has a larger diameter than injector 52. Injector 52 is preferably positioned within or near the lower end of draft tube. In this manner, the flow of fluid from injector 52 draws additional fluid from lower zone 37 into draft tube, as indicated by arrow 58. Fluid flows upward through draft tube 50 as indicated by arrow 60 and into the liquid phase above packing material 32. If desired, draft tube 50 may include a cover (not shown) positioned above said outlet of said draft tube so as to prevent solids in said digestion medium from falling into said outlet end.

The upward flow of fluid through draft tube 50 aids circulation within digester 12, increases agitation within the liquid phase, and enhances the flow of digesting biomass downward through the packing material. The velocity of the motive fluid exiting injector 52 is preferably sufficient to educt at least about 1 part educted fluid into draft tube 50 for each part motive fluid exiting injector 52 and more preferably sufficient to educt at least about 2 parts educted fluid for each part motive fluid. Alternatively, the velocity of the motive fluid exiting injector 52 may be at least about 0.01 m/s. In still further embodiments, diameter of injector 52 is less than 50% % of the diameter of updraft tube 50. In some embodiments, it may be desirable to inject a gas stream directly into draft tube 50 or to inject a gas stream into the fluid circulation system such that it exits into the draft tube via said injector 52.

In some embodiments, the outlet of draft tube 50 is positioned at at least 80% of the height of the reactor, which may or may not be below the surface 34 of the liquid slurry. Thus, digester 12 may be operated such that the level of the fluid phase in the reactor is above the outlet of draft tube 50, or such that the outlet of draft tube 50 is in headspace 35. It is preferred but not necessary that the outlet of draft tube 50 be above the top of mass of packing material 32; in some embodiments the outlet of draft tube 50 is within the packed zone.

After exiting draft tube 50, the liquid phase and slurry catalyst may contact the cellulosic biomass solids circulating above the packing material and may again migrate downward therethrough. Optionally, the slurry catalyst particulates conveyed via line 20 may be regenerated, if needed, while outside the digester 12. Further optionally, if insufficient slurry catalyst particulates are present, additional slurry catalyst particulates may be added to the continuous liquid phase in line 20 or 22.

### Downdraft System

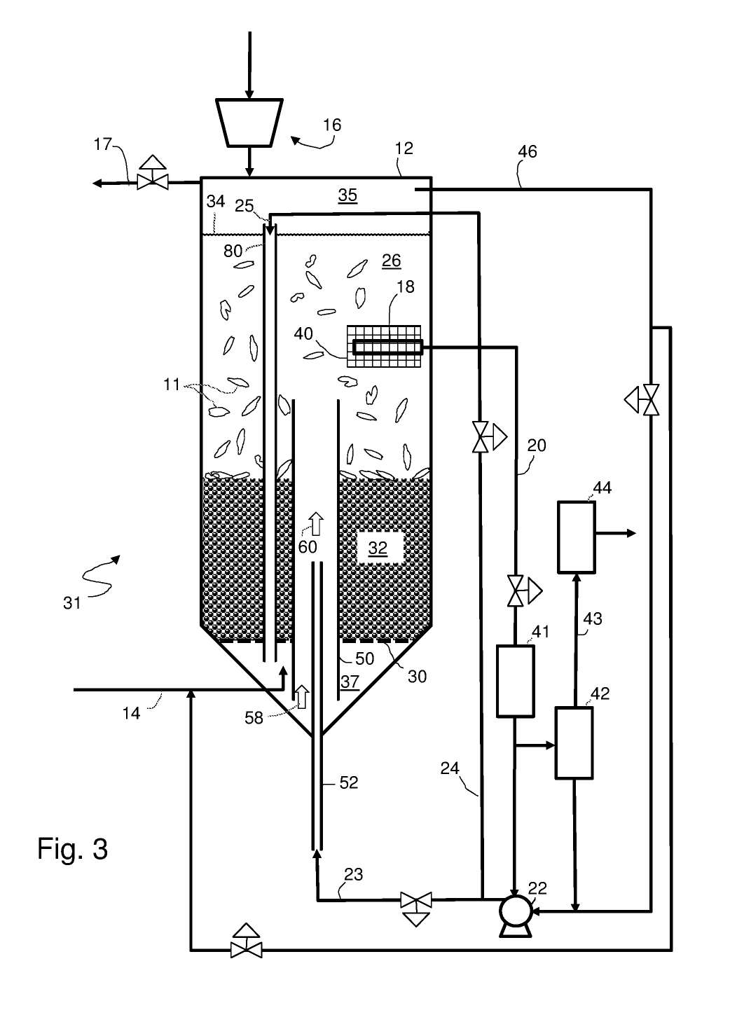

Referring now to FIG. 3, in a digester system 31 according to another embodiment of the invention, the upward flow in updraft tube 50 is complemented by a downdraft tube 80, which provides a downward flow. Specifically, by operating digester 12 such that a headspace 35 is provided, downdraft tube 80 can be positioned in the digester such that its inlet is in headspace 35 and its outlet is in the liquid slurry. The catalyst circulation system, which may include lines 20, 23, and 24, preferably includes a second fluid injector 25 positioned within downdraft tube 80 and preferably within the upper end of downdraft tube 80. Second fluid injector 25 is preferably the outlet of line 24 downstream of pump 22 and at higher pressure than the pressure in headspace 35. Thus, fluid flowing from second injector 25 draws gas from headspace 35 into the downdraft tube. Fluid from second injector 25 and gas entrained therewith flow out of downdraft tube 80 and are preferably discharged into lower zone 37.

Downdraft tube 80 can be used in conjunction with updraft tube 50 as described elsewhere herein, as shown in FIG. 3 or, if desired, can be used without an updraft tube. Similarly, digester 12 can be provided with a single downdraft tube 80, or with multiple downdraft tubes. In the latter case, pressurized fluid in line 24 can be manifolded to a plurality of injectors. The second end of downdraft tube 80 is preferably in lower zone 37, but may alternatively be within the mass of reactor packing material 32.

In preferred embodiments, the velocity of fluid exiting second injector 25 is at least 0.1 m/s. Alternatively, the velocity of the fluid exiting second injector 25 is preferably sufficient to educt at least about 1 part (by volume) of gas into draft tube 80 for each part liquid exiting injector 25 and more preferably sufficient to educt at least about 2 parts (by volume) of gas for each part liquid. In still further embodiments, the diameter of the second injector 25 is less than 25% of the diameter of downdraft tube 80. In alternative embodiments, the pressure within line 24 may be at least 10 kPa greater than the pressure in headspace 35.

If desired, the catalyst circulation system may also include a recycle fluid outlet that is within reactor 12 but not in either draft tube. In these embodiments, the outlet may be in headspace 35, in the liquid slurry 26, in the packing material 32, or in lower zone 37.

### Double Eductor Downdraft System

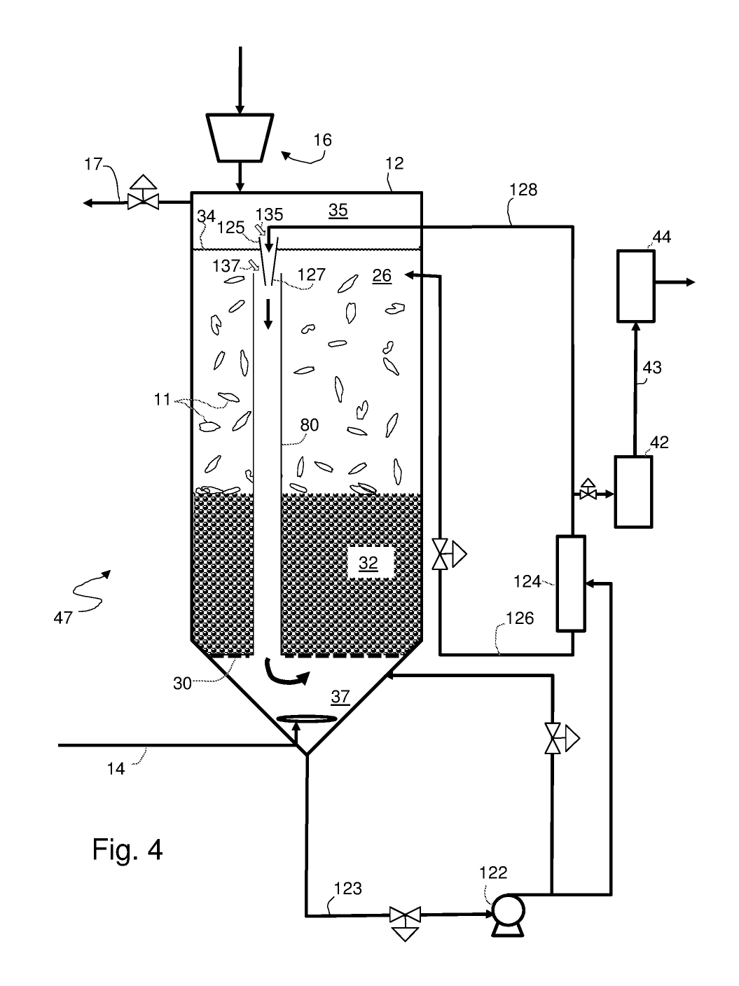

Referring now to FIG. 4, in a digester system 47 according to another embodiment of the invention, upward draft tube 50 may be eliminated and a series of eductors can be used to draw fluids from multiple regions in the top of reactor 12 to lower zone 37, thereby enhancing circulation within reactor 12. More specifically, slurry removed from the bottom of reactor 12 via line 123 can be pumped via a pump 122 to a hydroclone 124, gravity separator or other suitable type of separator, which separates the stream into a catalyst concentrate stream and a stream that is relatively low in solids. The catalyst concentrate stream exits the bottom of separator 124 via line 126 and is fed back into reactor 12 at a point that is preferably above the packed bed 32. If desired, the catalyst stream in line 126 may be fed into reactor 12 near the top of the liquid slurry 26. The low-solids stream exits separator 124 via line 128.

In certain preferred embodiments, between from about half up to all of the volume in line 123 is returned to reactor 12 via line 128. The outflow of line 128 is at a higher pressure than the fluid in the reactor and preferably serves as the motive fluid for a first eductor 125, drawing gas from headspace 35 into the fluid stream. The resulting gas/liquid stream is in turn preferably exits eductor 125 via a second injector 127 and serves as the motive force to draw fluid from liquid slurry 26, and more preferably near the top of slurry 26 into downdraft tube 80. It is generally desirable to prevent biomass solids from being dropped or drawn into eductor 125; if necessary a screen or other device (not shown) may be included for that purpose. Similarly, it is preferable to protect draft tube 80 from solids ingress, this can be accomplished by providing a mechanical screen or the like (not shown) for that purpose.

As in embodiments described above, a screen 30 supports the reactor packing material and prevents the biomass solids above a certain size from flowing into lower zone 37. Also as above, hydrogen may be sparged beneath screen 30, which helps prevent screen plugging. Alternatively hydrogen can be supplied at multiple locations within the reactor, as desired. As set out above, suitable gas distribution systems may include slotted distributors, manifolds, empty piping with an array of holes disposed thereon, sintered metal elements, collections of nozzles at a spacing effective to disperse a gas phase, other gas distribution manifolds, combinations thereof, and the like. It has been observed that upward-flowing gas bubbles can cause beneficial agitation of the biomass solids on the packing material. It is generally not desirable to include a gas phase in the stream that enters pump 122, so in some instances a gas separator (not shown) may be included between the bottom of the reactor and the pump inlet.

In this embodiment, the flow rate of fluids in draft tube 80 is preferably sufficient to carry the entrained gas bubbles below the packed ring section so that eductors 125, 127 provide gas recirculation, reducing or eliminating the need for a gas recycle compressor. As a result of fluid exiting downdraft tube 80, the pressure in lower zone 37 is greater than the pressure above the packed bed. This causes an upward flow of liquid and gas through the packing material and helps to prevent flooding of rings packed with wood, which would otherwise cause an undesirable buildup of gas in the bed. Upon exiting draft tube 80, the bubbles and liquid reverse direction and flow upward through bed 32.

While some slurry catalyst may recirculate via line 128, it is believed to be preferable to recycle the catalyst back to reactor 12 separately, such as via line 126. In addition, it has been found that, conventional pumps may not be sufficient for the functionality required of pump 122, in which case it may be necessary to make alternative provisions, either by using a specially-built pump or modifying the fluid stream so that it can be pumped by available equipment.

### 3-Eductor Digester/Reactor/Extractor

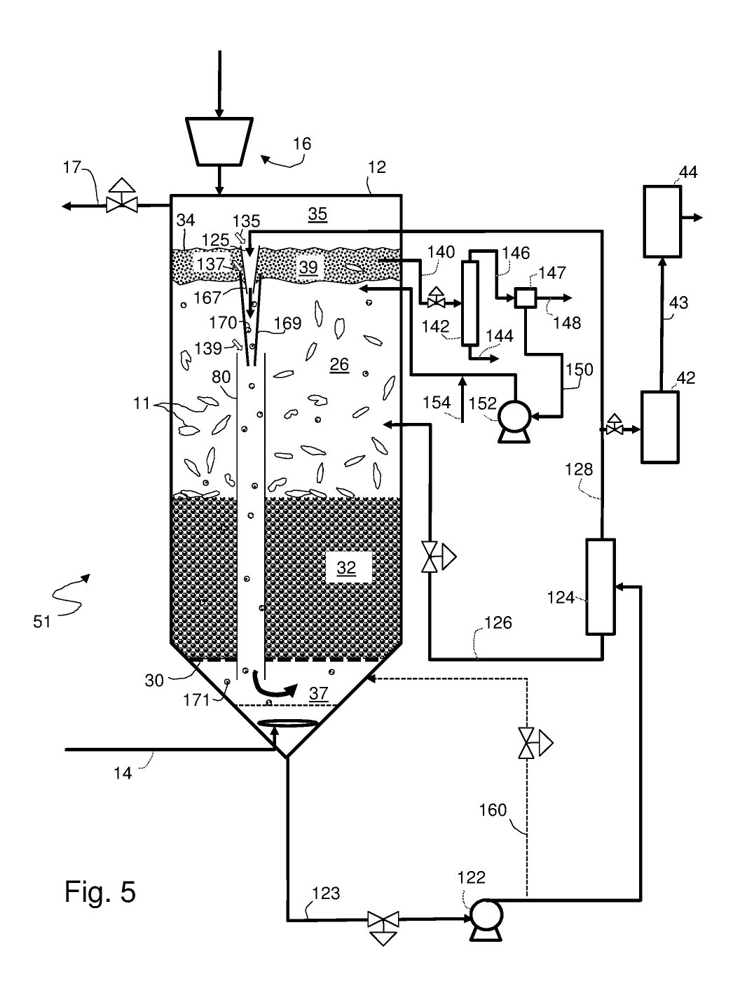

The digester-reactors described above produce alcohols (monox and diols), but not the aromatics needed to solubilize lignin. Aromatic solvents such as aromatic gasoline, diesel-type fractions, or toluene can be incorporated into the digester system if appropriate adjustments are made. Referring now to FIG. 5, in a digester system 51 according to another embodiment of the invention, an aromatic solvent is included in the reactor and is recycled as a solvent in combination with the produced alcohols to make the composite solvent. The system can be said to operate in two liquid phase regions.

As illustrated in FIG. 5, an organic layer 39 may be maintained at a desired level between the aqueous slurry 26, on which it floats, and the gas-filled headspace 35. The organic layer is preferably continuously removed from reactor 12 via a line 140 and flashed in a separator 142 to remove the lignin that would otherwise contribute to tar formation in the reactor. Specifically, lignin and asphaltenes leave the bottom of separator 142 via line 144, while the lighter organic fraction is taken off the top via line 146 and enters an accumulator 147, from which it may either be removed as intermediate coproduct via line 148 or returned to reactor 12 via line 150 and pump 152. If needed, make-up solvent can be added into line 150, as shown at 154.

According to this embodiment, aromatic solvent containing lipophilic longer chain diols, monox, plus phenols and some made THFA is mostly recycled, while some is diverted via line 148 and sent downstream to, for example, an acid condensation reaction so as to avoid buildup. The ratio of the amount recycled vs. liquid drawn off as intermediate product is preferably in the range of from about 0.5 parts recycle to 1 part intermediate product to about 10 parts recycle to 1 part intermediate product. Most typically, the recycle ratio will be between about 1 to 3 parts recycled per part of intermediate product withdrawn. The organic solvent will typically include some methanol, ethanol, and propanol; a portion of those alcohols will be removed from system the along with the solvent in line 148 but most will be recycled via line 150, thereby providing some additional monox alcohol solvent in the aromatic solvent (ArAlc solvent). Solvent composition is preferably controlled independently from lignin, which is rejected per pass.

As described above, slurry removed from the bottom of reactor 12 via line 123 can be pumped via a pump 122 to a hydroclone 124, gravity separator or other suitable type of separator, which separates the stream into a catalyst concentrate stream and a stream that is relatively low in solids. The catalyst concentrate stream exits the bottom of separator 124 via line 126 and can be fed back into reactor 12 at a point that is preferably above the packed bed 32 and may, if desired, be near the top of the liquid slurry 26. If desired, a stream of slurry 160 can optionally be separated from line 123 and returned to the bottom of reactor 12.

The low-solids stream exits separator 124 via line 128. In certain preferred embodiments, between half and all of the volume in line 123 is returned to reactor 12 via line 128. The outflow of line 128 is at a higher pressure than the fluid in the reactor and the fluid exiting a first fluid injector preferably serves as the motive fluid for a first eductor, drawing gas from headspace 35 into the fluid stream as shown at 135. The inlet orifice to eductor the first eductor may be configured to create fine gas bubbles (not shown), if desired. The resulting gas/liquid stream exits the first eductor via second injector or nozzle 167 and serves in turn as the motive force for a second eductor, which draws solvent-rich organic extractant from organic layer 39 into the fluid stream as shown at 137, forming organic droplets as shown at 170. The resulting gas/liquid/liquid stream leaves the second eductor via a third injector or nozzle 169 and serves in turn to draw fluid from liquid slurry 26 into downdraft tube 80 as shown at 139. The aqueous slurry drawn into draft tube 80 may include catalyst that was returned to reactor 12 via line 126, but if the catalyst is more dense than the aqueous phase, there is not likely to be much catalyst in the entrained liquid. If the catalyst is less dense than the aqueous phase, catalyst can be fed back at a point lower in the reactor.

It is generally desirable to prevent biomass solids from being dropped or drawn into the eductors or draft tube; if necessary screens or other devices (not shown) may be included for this purpose.

Fluid leaving first injector 125 in the first eductor aspirates H₂ gas into the aqueous recycle stream from headspace 35. The flow rate through the eductors and draft tube 80 is preferably sufficient to carry the gas, which can be both dissolved and bubbles, all the way to lower zone 37. Being less dense than water, the entrained gas bubbles (not shown) and organic solvent droplets (shown at 171) exiting draft tube 80 both flow upward through the packed bed and liquid slurry. With respect to the hydrogen, this can provide effective gas recycle and reduce or, more preferably, eliminate the need for a gas recycle compressor.

In addition, the dispersed organic liquid solvent droplets 171 exiting down draft tube 80 rise through the aqueous slurry and extract lignin while leaving the ethylene glycol and polyethylene glycol in the aqueous phase. EG and PG are poor solvents, and do not wish to recycle. Only longer diols and monox that can partition into the solvent, including the small amount of phenols and cyclic ethers that are generated, are extracted into the organic phase and build up in recycle. Thus, the organic phase 39 becomes enriched in the components desired and rejects those not desired (EG, PG). The lignin extracted into the organic phase is removed and rejected in separator 142.

The embodiment of FIG. 5 includes a second liquid phase in the digester. It is believed that extraction of products will occur both in the downdraft tube 80 and during the upward flow of organic droplets after they leave downdraft tube 80. The advantage of this is that only one solvent is needed in the digester, namely toluene or other aromatic solvent, which may be generated elsewhere in the process. The other, alcohol solvent component of the desired solvent blend is also made in situ.

It may be preferable in some instances to minimize foaming at the gas-liquid interface and the formation of an emulsion at the liquid-liquid interface. It is preferable to ensure that catalyst does not stray into the organic phase or it can be lost to lignin asphalt; this may be accomplished using a filter or screen below the liquid-liquid interface or by providing a magnetic field that keeps the catalyst in the aqueous phase.

The extent of hydrodeoxygenation (HDO) can be controlled via adjustments to catalyst concentration and temperature, or via use of a separate catalytic reactor to convert more of the polyoxygenated species to monooxygenate components, both of which can serve as solvents. Increased formation of monooxygenates such as ethanol and propanol from ethylene glycol and propylene glycol via an intensified or separate HDO step can simplify subsequent processing in acid condensation steps, which may prefer monoxygenates vs. diols to reduce tendency for coke formation. However, monooxygenates have higher vapor pressure than the polyols from which they are derived, and this can increase the required pressure for the HDO digestion and reaction step, in order to maintain a hydrogen partial pressure effective for the HDO reaction.

### Variations

It will be understood that, while the Figures show an updraft tube and/or a downdraft tube used in conjunction with reactor packing material, each of those reactor components can be used independently of the others. Similarly, updraft and downdraft tubes can each be used alone or in combination and provided singly or as a plurality of tubes. Similarly, upward and downward eductors and upward and downward draft tubes can be selected and combined in ways not explicitly disclosed. For example an upward eductor could be used in draft tube 50 of FIG. 1. Likewise, the catalyst separation and recirculation system 124, 126, 128 of FIG. 4 can replace the stinger and fluid circulation system 20, 23, 24 of FIG. 1 or vice versa.

Likewise, it will be understood that the various draft tubes or eductors that are described above and illustrated as being inside the reactor 12, can alternatively be provided outside of the reactor. Positioning draft tubes within the reactor has the advantage of avoiding the need for heat tracing or insulation and of allowing less rigorous specifications for the tube(s) and associate equipment. External equipment is more easily serviced, but requires additional equipment expense in the form of high pressure lines and thermal insulation. Internal eductors can be of the tank mix type and use a low pressure draft tube approach. Alternatively, but within the scope of the present invention, hard-piped eductors can used and can be internal or can be used with external hard piping lines. In any event, it will be understood that the various pipes, eductors, intakes, and discharges can each be positioned and/or provided multiply, in order to optimize cost, fluid flow, and reactor operation. For clarity, as used herein, “draft tube” refers to any conduit through which the fluid flow includes at least one entrained stream and “eductor” refers to a component that uses a first, relatively higher pressure fluid flowing through a constriction or injector to serve as a motive force that draws or entrains a second fluid into a fluid stream.

### Further Processing

For simplicity further processing of the components generated in the reactor are described below in terms of the embodiment shown in FIG. 1 but it will be understood that the principles set out below relate equally to all embodiments of the invention.

Referring again to FIG. 1, all or a portion of the continuous liquid phase exiting hydrothermal digestion unit 12 via line 20 may undergo further processing, either before being returned to digester 12, or for off-take. In some embodiments, a polishing reactor 41 may be positioned on line 20. Polishing reactor 41 may contain a catalyst capable of activating molecular hydrogen such that soluble carbohydrates in the continuous liquid phase may be further converted into an alcoholic component or the degree of oxygenation of the alcoholic component may be further decreased. For example, in some embodiments, a glycol may be converted into a monohydric alcohol in polishing reactor 41. The catalyst present in polishing reactor 41 may be the same or different than the slurry catalyst.

In further optional embodiments, all or a portion of the liquid in line 20 may be conveyed to a separations unit 42, where various operations may take place. In some embodiments, at least a portion of any water present in the continuous liquid phase may be removed in separations unit 42 before subsequent processing. In some embodiments, a phenolics liquid phase comprising at least a portion of the liquid phase may be separated from the liquid phase for further processing, or the viscosity of the phenolics liquid phase may be reduced. In some embodiments, the alcoholic component present in the liquid phase may be at least partially separated therefrom in separations unit 42. Optionally, at least a portion of the separated alcoholic component may be recycled to hydrothermal digestion unit 12 via recycle line 23, if desired.

Separations unit 42 may employ any liquid-liquid or liquid-solid separation technique known to one having ordinary skill in the art. In the interest of simplicity, the figures show a single line exiting separations unit 42, but it is to be recognized that depending on the type of separation being performed and the eventual destination of the component being separated, multiple lines may emanate from separations unit 42. A fluid exiting separations unit 42 may be returned to hydrothermal digestion unit 10 via line 23 or removed therefrom for further processing. It will be understood that the lines returning separated fluids and/or catalyst slurry to digester 12 may be configured other than as shown and may comprise multiple lines if desired.

The alcoholic component exiting separations unit 42 may be conveyed to reforming reactor 44 via line 43. Optionally, reaction products arising from lignin depolymerization (e.g., phenolic compounds) may also be conveyed to reforming reactor 44 along with the alcoholic component and/or methanol for further processing. In reforming reactor 44, a condensation reaction or other reforming reaction may take place. The reforming reaction taking place therein may be catalytic or non-catalytic. Although only one reforming reactor 44 has been depicted in FIG. 1, it is to be understood that any number of reforming reactors may be present.

In some embodiments, the present biomass conversion systems may further comprise a gas recirculation line 46 configured to increase gas circulation and turbulence in the digester. In some embodiments, the gas recirculation line may have its inlet in optional headspace 35. Recirculating a gas from the vertical fluid connection may present particular advantages in certain embodiments. For example, if liquid levels are properly maintained in the hydrothermal digestion unit such that a liquid does not back up into the gas inlet, the gas recirculation line may withdraw a gas (e.g., molecular hydrogen) from the digester without withdrawing a liquid therefrom. A gas distribution system that is kept largely free of liquid and solids may effectively channel and redistribute the gas phase from bottom to top of the biomass conversion system using the natural buoyancy of the gas phase.

In some embodiments, the biomass conversion systems may further comprise a biomass feed mechanism that is configured for addition of cellulosic biomass solids to the digester while it is in a pressurized state (e.g., at least about 30 bar). Inclusion of the biomass feed mechanism may allow cellulosic biomass solids to be continuously or semi-continuously fed to the hydrothermal digestion unit, thereby allowing hydrothermal digestion to take place in a continual manner by replenishing cellulosic biomass solids that have been digested to form soluble carbohydrates. Suitable biomass feed mechanisms are known. It is preferred to provide a system having the ability to introduce fresh cellulosic biomass solids to a pressurized hydrothermal digestion unit, so that biomass addition can be accomplished without depressurization and cooling of the hydrothermal digestion unit, which would significantly reduce the energy- and cost-efficiency of the biomass conversion process. As used herein, the term “continuous addition” and grammatical equivalents thereof will refer to a process in which cellulosic biomass solids are added to a hydrothermal digestion unit in an uninterrupted manner without fully depressurizing the hydrothermal digestion unit. As used herein, the term “semi-continuous addition” and grammatical equivalents thereof will refer to a discontinuous, but as-needed, addition of cellulosic biomass solids to a hydrothermal digestion unit without fully depressurizing the hydrothermal digestion unit. Some aspects of the techniques through which cellulosic biomass solids may be added continuously or semi-continuously to a pressurized hydrothermal digestion unit are discussed in more detail hereinbelow.

In some embodiments, cellulosic biomass solids being continuously or semi-continuously added to the hydrothermal digestion unit may be pressurized before being added to the hydrothermal digestion unit, particularly when the hydrothermal digestion unit is in a pressurized state. Pressurization of the cellulosic biomass solids from atmospheric pressure to a pressurized state may take place in one or more pressurization zones before addition of the cellulosic biomass solids to the hydrothermal digestion unit. Suitable pressurization zones that may be used for pressurizing and introducing cellulosic biomass solids to a pressurized hydrothermal digestion unit are described in more detail in commonly owned US20130152457 and US20130152458 and incorporated herein by reference in its entirety. Suitable pressurization zones described therein may include, for example, pressure vessels, pressurized screw feeders, and the like. In some embodiments, multiple pressurization zones may be connected in series to increase the pressure of the cellulosic biomass solids in a stepwise manner. Pressurization may take place via addition of a gas or a liquid to the pressurization zone. In some embodiments, a liquid being used for pressurization may comprise a fluid phase that is injected via injector 52.

In some embodiments, the present biomass conversion systems may further comprise a sump (not shown) at the lowermost point of lower zone 37. The sump may collect a fluid phase that has completed its downward progression through the hydrothermal digestion unit or that has been formed in conjunction with the hydrothermal digestion process. In some embodiments, a fluid phase collected in the sump may be recirculated in the hydrothermal digestion unit. Any fluid phase in the sump may be recirculated therefrom.

While reforming reactor 44 will, if present, typically contain a condensation reaction, it will be understood that additional reforming reactions contained therein may comprise any combination of further catalytic reduction reactions (e.g., hydrogenation reactions, hydrogenolysis reactions, hydrotreating reactions, and the like), further condensation reactions, isomerization reactions, desulfurization reactions, dehydration reactions, oligomerization reactions, alkylation reactions, and the like. Such transformations may be used to convert the initially produced soluble carbohydrates into a biofuel. Such biofuels may include, for example, gasoline hydrocarbons, diesel fuels, jet fuels, and the like. As used herein, the term “gasoline hydrocarbons” refers to substances comprising predominantly C₅-C₉ hydrocarbons and having a boiling point of 32° C. to about 204° C. More generally, any fuel blend meeting the requirements of ASTM D2887 may be classified as a gasoline hydrocarbon. Suitable gasoline hydrocarbons may include, for example, straight run gasoline, naphtha, fluidized or thermally catalytically cracked gasoline, VB gasoline, and coker gasoline. As used herein, the term “diesel fuel” refers to substances comprising paraffinic hydrocarbons and having a boiling point ranging between about 187° C. and about 417° C., which is suitable for use in a compression ignition engine. More generally, any fuel blend meeting the requirements of ASTM D975 may also be defined as a diesel fuel. As used herein, the term “jet fuel” refers to substances meeting the requirements of ASTM D1655. In some embodiments, jet fuels may comprise a kerosene-type fuel having substantially C₈-C₁₆ hydrocarbons (Jet A and Jet A-1 fuels). In other embodiments, jet fuels may comprise a wide-cut or naphtha-type fuel having substantially C₅-C₁₅ hydrocarbons present therein (Jet B fuels).

As discussed above, the cellulosic biomass solids may be introduced to the hydrothermal digestion unit separately from the liquid phase digestion medium and the cellulosic biomass solids. However, in alternative embodiments, the liquid phase digestion medium and slurry catalyst may be recirculated to the cellulosic biomass solids such that the cellulosic biomass solids, the liquid phase digestion medium, and the slurry catalyst are all introduced to the hydrothermal digestion unit at substantially the same time.

In some embodiments, the methods described herein may further comprise returning at least a portion of the liquid phase digestion medium and the slurry catalyst to the hydrothermal digestion unit. As discussed above, returning the liquid phase digestion medium and the slurry catalyst to the hydrothermal digestion unit may allow hydrothermal digestion to continue unabated and promote contact between the cellulosic biomass solids and the catalyst via fluid motion in the hydrothermal digestion unit.

### Reactions

Further discussion of the transformations that take place on the cellulosic biomass solids in the hydrothermal digestion unit and thereafter are now described in greater detail. In various embodiments, the alcoholic component derived from the cellulosic biomass solids may be formed by a catalytic reduction reaction of soluble carbohydrates, where the soluble carbohydrates are derived from the cellulosic biomass solids. As described above, the methods and systems set forth herein can help promote adequate distribution of the slurry catalyst and the molecular hydrogen throughout the cellulosic biomass solids such that the catalytic reduction reaction can more effectively take place.

In some embodiments, the catalytic reduction reaction used to produce the alcoholic component may take place at a temperature ranging between about 110° C. and about 300° C., or between about 170° C. and about 300° C., or between about 180° C. and about 290° C., or between about 150° C. and about 250° C. In some embodiments, the catalytic reduction reaction used to produce the alcoholic component may take place at a pH ranging between about 7 and about 13, or between about 10 and about 12. In other embodiments, the catalytic reduction reaction may take place under acidic conditions, such as at a pH of about 5 to about 7. Acids, bases, and buffers may be introduced as necessary to achieve a desired pH level. In some embodiments, the catalytic reduction reaction may be conducted under a hydrogen partial pressure ranging between about 1 bar (absolute) and about 150 bar, or between about 15 bar and about 140 bar, or between about 30 bar and about 130 bar, or between about 50 bar and about 110 bar.

In various embodiments, the liquid phase digestion medium in which the hydrothermal digestion and catalytic reduction reaction are conducted may comprise an organic solvent and water. Although any organic solvent that is at least partially miscible with water may be used as a digestion solvent, particularly advantageous organic solvents are those that can be directly converted into fuel blends and other materials without being separated from the alcoholic component being produced from the cellulosic biomass solids. That is, particularly advantageous organic solvents are those that may be co-processed along with the alcoholic component during downstream reforming reactions into fuel blends and other materials. Suitable organic solvents in this regard may include, for example, ethanol, ethylene glycol, propylene glycol, glycerol, and any combination thereof.

In some embodiments, the liquid phase digestion medium may further comprise a small amount of a monohydric alcohol. The presence of at least some monohydric alcohols in the liquid phase digestion medium may desirably enhance the hydrothermal digestion and/or the catalytic reduction reactions being conducted therein. For example, inclusion of about 1% to about 5% by weight monohydric alcohols in the liquid phase digestion medium may desirably maintain catalyst activity due to a surface cleaning effect. Monohydric alcohols present in the digestion solvent may arise from any source. In some embodiments, the monohydric alcohols may be formed via the in situ catalytic reduction reaction process being conducted therein. In some or other embodiments, the monohydric alcohols may be formed during further chemical transformations of the initially formed alcoholic component. In still other embodiments, the monohydric alcohols may be sourced from an external feed that is in flow communication with the cellulosic biomass solids.

In some embodiments, the liquid phase digestion medium may comprise between about 1% water and about 99% water. Although higher percentages of water may be more favorable from an environmental standpoint, higher quantities of organic solvent may more effectively promote hydrothermal digestion due to the organic solvent's greater propensity to solubilize carbohydrates and promote catalytic reduction of the soluble carbohydrates. In some embodiments, the liquid phase digestion medium may comprise about 90% or less water by weight. In other embodiments, the liquid phase digestion medium may comprise about 80% or less water by weight, or about 70% or less water by weight, or about 60% or less water by weight, or about 50% or less water by weight, or about 40% or less water by weight, or about 30% or less water by weight, or about 20% or less water by weight, or about 10% or less water by weight, or about 5% or less water by weight.

In some embodiments, catalysts capable of activating molecular hydrogen and conducting a catalytic reduction reaction may comprise a metal such as, for example, Cr, Mo, W, Re, Mn, Cu, Cd, Fe, Co, Ni, Pt, Pd, Rh, Ru, Ir, Os, and alloys or any combination thereof, either alone or with promoters such as Au, Ag, Cr, Zn, Mn, Sn, Bi, B, O, and alloys or any combination thereof. In some embodiments, the catalysts and promoters may allow for hydrogenation and hydrogenolysis reactions to occur at the same time or in succession of one another. In some embodiments, such catalysts may also comprise a carbonaceous pyropolymer catalyst containing transition metals (e.g., Cr, Mo, W, Re, Mn, Cu, and Cd) or Group VIII metals (e.g., Fe, Co, Ni, Pt, Pd, Rh, Ru, Ir, and Os). In some embodiments, the foregoing catalysts may be combined with an alkaline earth metal oxide or adhered to a catalytically active support. In some or other embodiments, the catalyst capable of activating molecular hydrogen may be deposited on a catalyst support that is not itself catalytically active.

In some embodiments, the catalyst that is capable of activating molecular hydrogen may comprise a slurry catalyst. In some embodiments, the slurry catalyst may comprise a poison-tolerant catalyst. As used herein the term “poison-tolerant catalyst” refers to a catalyst that is capable of activating molecular hydrogen without needing to be regenerated or replaced due to low catalytic activity for at least about 12 hours of continuous operation. Use of a poison-tolerant catalyst may be particularly desirable when reacting soluble carbohydrates derived from cellulosic biomass solids that have not had catalyst poisons removed therefrom. Catalysts that are not poison tolerant may also be used to achieve a similar result, but they may need to be regenerated or replaced more frequently than does a poison-tolerant catalyst.

In some embodiments, suitable poison-tolerant catalysts may include, for example, sulfided catalysts. In some or other embodiments, nitrided catalysts may be used as poison-tolerant catalysts. Sulfided catalysts suitable for activating molecular hydrogen are described in commonly owned US20120317872 and US20130109896, each of which is incorporated herein by reference in its entirety. Sulfiding may take place by treating the catalyst with hydrogen sulfide or an alternative sulfiding agent, optionally while the catalyst is disposed on a solid support. In more particular embodiments, the poison-tolerant catalyst may comprise a sulfided cobalt-molybdate catalyst, such as a catalyst comprising about 1-10 wt. % cobalt oxide and up to about 30 wt. % molybdenum trioxide. In other embodiments, catalysts containing Pt or Pd may also be effective poison-tolerant catalysts for use in the techniques described herein. When mediating in situ catalytic reduction reaction processes, sulfided catalysts may be particularly well suited to form reaction products comprising a substantial fraction of glycols (e.g., C₂-C₆ glycols) without producing excessive amounts of the corresponding monohydric alcohols. Although poison-tolerant catalysts, particularly sulfided catalysts, may be well suited for forming glycols from soluble carbohydrates, it is to be recognized that other types of catalysts, which may not necessarily be poison-tolerant, may also be used to achieve a like result in alternative embodiments. As will be recognized by one having ordinary skill in the art, various reaction parameters (e.g., temperature, pressure, catalyst composition, introduction of other components, and the like) may be modified to favor the formation of a desired reaction product. Given the benefit of the present disclosure, one having ordinary skill in the art will be able to alter various reaction parameters to change the product distribution obtained from a particular catalyst and set of reactants.

In some embodiments, slurry catalysts suitable for use in the methods described herein may be sulfided by dispersing a slurry catalyst in a fluid phase and adding a sulfiding agent thereto. Suitable sulfiding agents may include, for example, organic sulfoxides (e.g., dimethyl sulfoxide), hydrogen sulfide, salts of hydrogen sulfide (e.g., NaSH), and the like. In some embodiments, the slurry catalyst may be concentrated in the fluid phase after sulfiding, and the concentrated slurry may then be distributed in the cellulosic biomass solids using fluid flow. Illustrative techniques for catalyst sulfiding that may be used in conjunction with the methods described herein are described in U.S. Patent Application Publication No. 20100236988 and incorporated herein by reference in its entirety.

In various embodiments, slurry catalysts used in conjunction with the methods described herein may have a particulate size of about 250 microns or less. In some embodiments, the slurry catalyst may have a particulate size of about 100 microns or less, or about 10 microns or less. In some embodiments, the minimum particulate size of the slurry catalyst may be about 1 micron. In some embodiments, the slurry catalyst may comprise catalyst fines in the processes described herein. As used herein, the term “catalyst fines” refers to solid catalysts having a nominal particulate size of about 100 microns or less. Catalyst fines may be generated from catalyst production processes, for example, during extrusion of solid catalysts. Catalyst fines may also be produced by grinding larger catalyst solids or during regeneration of catalyst solids. Suitable methods for producing catalyst fines are described in U.S. Pat. Nos. 6,030,915 and 6,127,299, each of which is incorporated herein by reference in its entirety. In some instances, catalyst fines may be intentionally removed from a solid catalyst production run, since they may be difficult to sequester in some catalytic processes. Techniques for removing catalyst fines from larger catalyst solids may include, for example, sieving or like size separation processes. When conducting in situ catalytic reduction reaction processes, such as those described herein, catalyst fines may be particularly well suited, since they can be easily fluidized and distributed in the interstitial pore space of the digesting cellulosic biomass solids.

Catalysts that are not particularly poison-tolerant may also be used in conjunction with the techniques described herein. Such catalysts may include, for example, Ru, Pt, Pd, or compounds thereof disposed on a solid support such as, for example, Ru on titanium dioxide or Ru on carbon. Although such catalysts may not have particular poison tolerance, they may be regenerable, such as through exposure of the catalyst to water at elevated temperatures, which may be in either a subcritical state or a supercritical state.

In some embodiments, the catalysts used in conjunction with the processes described herein may be operable to generate molecular hydrogen. For example, in some embodiments, catalysts suitable for aqueous phase reforming (i.e., APR catalysts) may be used. Suitable APR catalysts may include, for example, catalysts comprising Pt, Pd, Ru, Ni, Co, or other Group VIII metals alloyed or modified with Re, Mo, Sn, or other metals.

In some embodiments, the alcoholic component formed from the cellulosic biomass solids may be further reformed into a biofuel. Reforming the alcoholic component into a biofuel or other material may comprise any combination and sequence of further hydrogenolysis reactions and/or hydrogenation reactions, condensation reactions, isomerization reactions, oligomerization reactions, hydrotreating reactions, alkylation reactions, dehydration reactions, desulfurization reactions, and the like. The subsequent reforming reactions may be catalytic or non-catalytic. In some embodiments, an initial operation of downstream reforming may comprise a condensation reaction, often conducted in the presence of a condensation catalyst, in which the alcoholic component or a product derived therefrom is condensed with another molecule to form a higher molecular weight compound. As used herein, the term “condensation reaction” will refer to a chemical transformation in which two or more molecules are coupled with one another to form a carbon-carbon bond in a higher molecular weight compound, usually accompanied by the loss of a small molecule such as water or an alcohol. An illustrative condensation reaction is the Aldol condensation reaction, which will be familiar to one having ordinary skill in the art. Additional disclosure regarding condensation reactions and catalysts suitable for promoting condensation reactions is provided hereinbelow.

In some embodiments, methods described herein may further comprise performing a condensation reaction on the alcoholic component or a product derived therefrom. In various embodiments, the condensation reaction may take place at a temperature ranging between about 5° C. and about 500° C. The condensation reaction may take place in a condensed phase (e.g., a liquor phase) or in a vapor phase. For condensation reactions taking place in a vapor phase, the temperature may range between about 75° C. and about 500° C., or between about 125° C. and about 450° C. For condensation reactions taking place in a condensed phase, the temperature may range between about 5° C. and about 475° C., or between about 15° C. and about 300° C., or between about 20° C. and about 250° C.

In various embodiments, the higher molecular weight compound produced by the condensation reaction may comprise C₄₊ hydrocarbons. In some or other embodiments, the higher molecular weight compound produced by the condensation reaction may comprise C₆₊ hydrocarbons. In some embodiments, the higher molecular weight compound produced by the condensation reaction may comprise C₄-C₃₀ hydrocarbons. In some embodiments, the higher molecular weight compound produced by the condensation reaction may comprise C₆-C₃₀ hydrocarbons. In still other embodiments, the higher molecular weight compound produced by the condensation reaction may comprise C₄-C₂₄ hydrocarbons, or C₆-C₂₄ hydrocarbons, or C₄-C₁₈ hydrocarbons, or C₆-C₁₈ hydrocarbons, or C₄-C₁₂ hydrocarbons, or C₆-C₁₂ hydrocarbons. As used herein, the term “hydrocarbons” refers to compounds containing both carbon and hydrogen without reference to other elements that may be present. Thus, heteroatom-substituted compounds are also described herein by the term “hydrocarbons.”

The particular composition of the higher molecular weight compound produced by the condensation reaction may vary depending on the catalyst(s) and temperatures used for both the catalytic reduction reaction and the condensation reaction, as well as other parameters such as pressure. For example, in some embodiments, the product of the condensation reaction may comprise C₄₊ alcohols and/or ketones that are produced concurrently with or in lieu of C₄₊ hydrocarbons. In some embodiments, the C₄₊ hydrocarbons produced by the condensation reaction may contain various olefins in addition to alkanes of various sizes, typically branched alkanes. In still other embodiments, the C₄₊ hydrocarbons produced by the condensation reaction may also comprise cyclic hydrocarbons and/or aromatic compounds. In some embodiments, the higher molecular weight compound produced by the condensation reaction may be further subjected to a catalytic reduction reaction to transform a carbonyl functionality therein to an alcohol and/or a hydrocarbon and to convert olefins into alkanes.

Exemplary compounds that may be produced by a condensation reaction include, for example, C₄₊ alkanes, C₄₊ alkenes, C₅₊ cycloalkanes, C₅₊ cycloalkenes, aryls, fused aryls, C₄₊ alcohols, C₄₊ ketones, and mixtures thereof. The C₄₊ alkanes and C₄₊ alkenes may range from 4 to about 30 carbon atoms (i.e. C₄-C₃₀ alkanes and C₄-C₃₀ alkenes) and may be branched or straight chain alkanes or alkenes. The C₄₊ alkanes and C₄₊ alkenes may also include fractions of C₇-C₁₄, C₁₂-C₂₄ alkanes and alkenes, respectively, with the C₇-C₁₄ fraction directed to jet fuel blends, and the C₁₂-C₂₄ fraction directed to diesel fuel blends and other industrial applications. Examples of various C₄₊ alkanes and C₄₊ alkenes that may be produced by the condensation reaction include, without limitation, butane, butene, pentane, pentene, 2-methylbutane, hexane, hexene, 2-methylpentane, 3-methylpentane, 2,2-dimethylbutane, 2,3-dimethylbutane, heptane, heptene, octane, octene, 2,2,4,-trimethylpentane, 2,3-dimethylhexane, 2,3,4-trimethylpentane, 2,3-dimethylpentane, nonane, nonene, decane, decene, undecane, undecene, dodecane, dodecene, tridecane, tridecene, tetradecane, tetradecene, pentadecane, pentadecene, hexadecane, hexadecene, heptyldecane, heptyldecene, octyldecane, octyldecene, nonyldecane, nonyldecene, eicosane, eicosene, uneicosane, uneicosene, doeicosane, doeicosene, trieicosane, trieicosene, tetraeicosane, tetraeicosene, and isomers thereof.

The C₅₊ cycloalkanes and C₅₊ cycloalkenes may have from 5 to about 30 carbon atoms and may be unsubstituted, mono-substituted or multi-substituted. In the case of mono-substituted and multi-substituted compounds, the substituted group may include a branched C₃₊ alkyl, a straight chain C₁₊ alkyl, a branched C₃₊ alkylene, a straight chain C₁₊ alkylene, a straight chain C₂₊ alkylene, an aryl group, or a combination thereof. In some embodiments, at least one of the substituted groups may include a branched C₃-C₁₂ alkyl, a straight chain C₁-C₁₂ alkyl, a branched C₃-C₁₂ alkylene, a straight chain C₁-C₁₂ alkylene, a straight chain C₂-C₁₂ alkylene, an aryl group, or a combination thereof. In yet other embodiments, at least one of the substituted groups may include a branched C₃-C₄ alkyl, a straight chain C₁-C₄ alkyl, a branched C₃-C₄ alkylene, a straight chain C₁-C₄ alkylene, a straight chain C₂-C₄ alkylene, an aryl group, or any combination thereof. Examples of C₅₊ cycloalkanes and C₅₊ cycloalkenes that may be produced by the condensation reaction include, without limitation, cyclopentane, cyclopentene, cyclohexane, cyclohexene, methylcyclopentane, methylcyclopentene, ethylcyclopentane, ethylcyclopentene, ethylcyclohexane, ethylcyclohexene, and isomers thereof.

The moderate fractions of the condensation reaction, such as C₇-C₁₄, may be separated for jet fuel, while heavier fractions, such as C₁₂-C₂₄, may be separated for diesel use. The heaviest fractions may be used as lubricants or cracked to produce additional gasoline and/or diesel fractions. The C₄₊ compounds may also find use as industrial chemicals, whether as an intermediate or an end product. For example, the aryl compounds toluene, xylene, ethylbenzene, para-xylene, meta-xylene, and ortho-xylene may find use as chemical intermediates for the production of plastics and other products. Meanwhile, C₉ aromatic compounds and fused aryl compounds, such as naphthalene, anthracene, tetrahydronaphthalene, and decahydronaphthalene, may find use as solvents or additives in industrial processes.

In some embodiments, a single catalyst may mediate the transformation of the alcoholic component into a form suitable for undergoing a condensation reaction as well as mediating the condensation reaction itself. In other embodiments, a first catalyst may be used to mediate the transformation of the alcoholic component into a form suitable for undergoing a condensation reaction, and a second catalyst may be used to mediate the condensation reaction. Unless otherwise specified, it is to be understood that reference herein to a condensation reaction and condensation catalyst refers to either type of condensation process. Further disclosure of suitable condensation catalysts now follows.

In some embodiments, a single catalyst may be used to form a higher molecular weight compound via a condensation reaction. Without being bound by any theory or mechanism, it is believed that such catalysts may mediate an initial dehydrogenation of the alcoholic component, followed by a condensation reaction of the dehydrogenated alcoholic component. Zeolite catalysts are one type of catalyst suitable for directly converting alcohols to condensation products in such a manner. A particularly suitable zeolite catalyst in this regard may be ZSM-5, although other zeolite catalysts may also be suitable.

In some embodiments, two catalysts may be used to form a higher molecular weight compound via a condensation reaction. Without being bound by any theory or mechanism, it is believed that the first catalyst may mediate an initial dehydrogenation of the alcoholic component, and the second catalyst may mediate a condensation reaction of the dehydrogenated alcoholic component. Like the single-catalyst embodiments discussed previously above, in some embodiments, zeolite catalysts may be used as either the first catalyst or the second catalyst. Again, a particularly suitable zeolite catalyst in this regard may be ZSM-5, although other zeolite catalysts may also be suitable.

Various catalytic processes may be used to form higher molecular weight compounds by a condensation reaction. In some embodiments, the catalyst used for mediating a condensation reaction may comprise a basic site, or both an acidic site and a basic site. Catalysts comprising both an acidic site and a basic site will be referred to herein as multi-functional catalysts. In some or other embodiments, a catalyst used for mediating a condensation reaction may comprise one or more metal atoms. Any of the condensation catalysts may also optionally be disposed on a solid support, if desired.

In some embodiments, the condensation catalyst may comprise a basic catalyst comprising Li, Na, K, Cs, B, Rb, Mg, Ca, Sr, Si, Ba, Al, Zn, Ce, La, Y, Sc, Y, Zr, Ti, hydrotalcite, zinc-aluminate, phosphate, base-treated aluminosilicate zeolite, a basic resin, basic nitride, alloys or any combination thereof. In some embodiments, the basic catalyst may also comprise an oxide of Ti, Zr, V, Nb, Ta, Mo, Cr, W, Mn, Re, Al, Ga, In, Co, Ni, Si, Cu, Zn, Sn, Cd, Mg, P, Fe, or any combination thereof. In some embodiments, the basic catalyst may comprise a mixed-oxide basic catalyst. Suitable mixed-oxide basic catalysts may comprise, for example, Si—Mg—O, Mg—Ti—O, Y—Mg—O, Y—Zr—O, Ti—Zr—O, Ce—Zr—O, Ce—Mg—O, Ca—Zr—O, La—Zr—O, B—Zr—O, La—Ti—O, B—Ti—O, and any combination thereof. In some embodiments, the condensation catalyst may further include a metal or alloys comprising metals such as, for example, Cu, Ag, Au, Pt, Ni, Fe, Co, Ru, Zn, Cd, Ga, In, Rh, Pd, Ir, Re, Mn, Cr, Mo, W, Sn, Bi, Pb, Os, alloys and combinations thereof. Use of metals in the condensation catalyst may be desirable when a dehydrogenation reaction is to be carried out in concert with the condensation reaction. Basic resins may include resins that exhibit basic functionality. The basic catalyst may be self-supporting or adhered to a support containing a material such as, for example, carbon, silica, alumina, zirconia, titania, vanadia, ceria, nitride, boron nitride, a heteropolyacid, alloys and mixtures thereof.

In some embodiments, the condensation catalyst may comprise a hydrotalcite material derived from a combination of MgO and Al₂O₃. In some embodiments, the condensation catalyst may comprise a zinc aluminate spinel formed from a combination of ZnO and Al₂O₃. In still other embodiments, the condensation catalyst may comprise a combination of ZnO, Al₂O₃, and CuO. Each of these materials may also contain an additional metal or alloy, including those more generally referenced above for basic condensation catalysts. In more particular embodiments, the additional metal or alloy may comprise a Group 10 metal such Pd, Pt, or any combination thereof.

In some embodiments, the condensation catalyst may comprise a basic catalyst comprising a metal oxide containing, for example, Cu, Ni, Zn, V, Zr, or any mixture thereof. In some or other embodiments, the condensation catalyst may comprise a zinc aluminate containing, for example, Pt, Pd, Cu, Ni, or any mixture thereof.

In some embodiments, the condensation catalyst may comprise a multi-functional catalyst having both an acidic functionality and a basic functionality. Such condensation catalysts may comprise a hydrotalcite, a zinc-aluminate, a phosphate, Li, Na, K, Cs, B, Rb, Mg, Si, Ca, Sr, Ba, Al, Ce, La, Sc, Y, Zr, Ti, Zn, Cr, or any combination thereof. In further embodiments, the multi-functional catalyst may also include one or more oxides from the group of Ti, Zr, V, Nb, Ta, Mo, Cr, W, Mn, Re, Al, Ga, In, Fe, Co, Ir, Ni, Si, Cu, Zn, Sn, Cd, P, and any combination thereof. In some embodiments, the multi-functional catalyst may include a metal such as, for example, Cu, Ag, Au, Pt, Ni, Fe, Co, Ru, Zn, Cd, Ga, In, Rh, Pd, Ir, Re, Mn, Cr, Mo, W, Sn, Os, alloys or combinations thereof. The basic catalyst may be self-supporting or adhered to a support containing a material such as, for example, carbon, silica, alumina, zirconia, titania, vanadia, ceria, nitride, boron nitride, a heteropolyacid, alloys and mixtures thereof.

In some embodiments, the condensation catalyst may comprise a metal oxide containing Pd, Pt, Cu or Ni. In still other embodiments, the condensation catalyst may comprise an aluminate or a zirconium metal oxide containing Mg and Cu, Pt, Pd or Ni. In still other embodiments, a multi-functional catalyst may comprise a hydroxyapatite (HAP) combined with one or more of the above metals.

In some embodiments, the condensation catalyst may also include a zeolite and other microporous supports that contain Group IA compounds, such as Li, Na, K, Cs and Rb. Preferably, the Group IA material may be present in an amount less than that required to neutralize the acidic nature of the support. A metal function may also be provided by the addition of group VIIIB metals, or Cu, Ga, In, Zn or Sn. In some embodiments, the condensation catalyst may be derived from the combination of MgO and Al₂O₃ to form a hydrotalcite material. Another condensation catalyst may comprise a combination of MgO and ZrO₂, or a combination of ZnO and Al₂O₃. Each of these materials may also contain an additional metal function provided by copper or a Group VIIIB metal, such as Ni, Pd, Pt, or combinations of the foregoing.

The condensation reaction mediated by the condensation catalyst may be carried out in any reactor of suitable design, including continuous-flow, batch, semi-batch or multi-system reactors, without limitation as to design, size, geometry, flow rates, and the like. The reactor system may also use a fluidized catalytic bed system, a swing bed system, fixed bed system, a moving bed system, or a combination of the above. In some embodiments, bi-phasic (e.g., liquid-liquid) and tri-phasic (e.g., liquid-liquid-solid) reactors may be used to carry out the condensation reaction.

In some embodiments, an acid catalyst may be used to optionally dehydrate at least a portion of the reaction product. Suitable acid catalysts for use in the dehydration reaction may include, but are not limited to, mineral acids (e.g., HCl, H₂SO₄), solid acids (e.g., zeolites, ion-exchange resins) and acid salts (e.g., LaCl₃). Additional acid catalysts may include, without limitation, zeolites, carbides, nitrides, zirconia, alumina, silica, aluminosilicates, phosphates, titanium oxides, zinc oxides, vanadium oxides, lanthanum oxides, yttrium oxides, scandium oxides, magnesium oxides, cerium oxides, barium oxides, calcium oxides, hydroxides, heteropolyacids, inorganic acids, acid modified resins, base modified resins, and any combination thereof. In some embodiments, the dehydration catalyst may also include a modifier. Suitable modifiers may include, for example, La, Y, Sc, P, B, Bi, Li, Na, K, Rb, Cs, Mg, Ca, Sr, Ba, and any combination thereof. The modifiers may be useful, inter alia, to carry out a concerted hydrogenation/dehydrogenation reaction with the dehydration reaction. In some embodiments, the dehydration catalyst may also include a metal. Suitable metals may include, for example, Cu, Ag, Au, Pt, Ni, Fe, Co, Ru, Zn, Cd, Ga, In, Rh, Pd, Ir, Re, Mn, Cr, Mo, W, Sn, Os, alloys, and any combination thereof. The dehydration catalyst may be self supporting, supported on an inert support or resin, or it may be dissolved in a fluid.

Various operations may optionally be performed on the alcoholic component prior to conducting a condensation reaction. In addition, various operations may optionally be performed on a fluid phase containing the alcoholic component, thereby further transforming the alcoholic component or placing the alcoholic component in a form more suitable for taking part in a condensation reaction. These optional operations are now described in more detail below.

As described above, one or more liquid phases may be present when digesting cellulosic biomass solids. Particularly when cellulosic biomass solids are fed continuously or semi-continuously to the hydrothermal digestion unit, digestion of the cellulosic biomass solids may produce multiple liquid phases in the hydrothermal digestion unit. The liquid phases may be immiscible with one another, or they may be at least partially miscible with one another. In some embodiments, the one or more liquid phases may comprise a phenolics liquid phase comprising lignin or a product formed therefrom, an aqueous phase comprising the alcoholic component, a light organics phase, or any combination thereof. The alcoholic component being produced from the cellulosic biomass solids may be partitioned between the one or more liquid phases, or the alcoholic component may be located substantially in a single liquid phase. For example, the alcoholic component being produced from the cellulosic biomass solids may be located predominantly in an aqueous phase (e.g., an aqueous phase digestion solvent), although minor amounts of the alcoholic component may be partitioned to the phenolics liquid phase or a light organics phase. In various embodiments, the slurry catalyst may accumulate in the phenolics liquid phase as it forms, thereby complicating the return of the slurry catalyst to the cellulosic biomass solids in the manner described above. Alternative configurations for distributing slurry catalyst particulates in the cellulosic biomass solids when excessive catalyst accumulation in the phenolics liquid phase has occurred are described hereinafter.

Accumulation of the slurry catalyst in the phenolics liquid phase may, in some embodiments, be addressed by conveying this phase and the accumulated slurry catalyst therein to the same location where a liquid phase digestion medium is being contacted with cellulosic biomass solids. The liquid phase digestion medium and the phenolics liquid phase may be conveyed to the cellulosic biomass solids together or separately. Thusly, either the liquid phase digestion medium and/or the phenolics liquid phase may motively return the slurry catalyst back to the cellulosic biomass solids such that continued stabilization of soluble carbohydrates may take place. In some embodiments, at least a portion of the lignin in the phenolics liquid phase may be depolymerized before or while conveying the phenolics liquid phase for redistribution of the slurry catalyst. At least partial depolymerization of the lignin in the phenolics liquid phase may reduce the viscosity of this phase and make it easier to convey. Lignin depolymerization may take place chemically by hydrolyzing the lignin (e.g., with a base) or thermally by heating the lignin to a temperature of at least about 250° C. in the presence of molecular hydrogen and the slurry catalyst. Further details regarding lignin depolymerization and the use of viscosity monitoring as a means of process control are described in commonly owned U.S. Patent Application 61/720,765, filed Oct. 31, 2012 and incorporated herein by reference in its entirety.

After forming the alcoholic component from the cellulosic biomass solids, at least a portion of the alcoholic component may be separated from the cellulosic biomass solids and further processed by performing a condensation reaction thereon, as generally described above. Processing of the alcoholic component that has partitioned between various liquid phases may take place with the phases separated from one another, or with the liquid phases mixed together. For example, in some embodiments, the alcoholic component in a liquid phase digestion medium may be processed separately from a light organics phase. In other embodiments, the light organics phase may be processed concurrently with the liquid phase digestion medium.

Optionally, the liquid phase digestion medium containing the alcoholic component may be subjected to a second catalytic reduction reaction external to the cellulosic biomass solids, if needed, for example, to increase the amount of soluble carbohydrates that are converted into the alcoholic component and/or to further reduce the degree of oxygenation of the alcoholic components that are formed. For example, in some embodiments, a glycol or more highly oxygenated alcohol may be transformed into a monohydric alcohol by performing a second catalytic reduction reaction. The choice of whether to perform a condensation reaction on a monohydric alcohol or a glycol may be based on a number of factors, as discussed in more detail below, and each approach may present particular advantages.

In some embodiments, a glycol produced from the cellulosic biomass solids may be fed to the condensation catalyst. Although glycols may be prone to coking when used in conjunction with condensation catalysts, particularly zeolite catalysts, the present inventors found the degree of coking to be manageable in the production of higher molecular weight compounds. Approaches for producing glycols from cellulosic biomass solids and feeding the glycols to a condensation catalyst are described in commonly owned U.S. patent application Ser. No. 14/067,428, filed Oct. 30, 2013 and incorporated herein by reference in its entirety. A primary advantage of feeding glycols to a condensation catalyst is that removal of water from glycols is considerably easier than from monohydric alcohols. Excessive water exposure can be particularly detrimental for zeolite catalysts and shorten their lifetime. Although monohydric alcohols are typically a preferred substrate for zeolite catalysts, they may be difficult to prepare in dried form due to azeotrope formation with water. Glycols, in contrast, are not believed to readily form binary azeotropes with water and may be produced in dried form by distillation.

In some embodiments, a dried alcoholic component, particularly a dried glycol, may be produced from cellulosic biomass solids and fed to a condensation catalyst. As used herein, the term “dried alcoholic component” refers to a fluid phase containing an alcoholic component that has had a least a portion of the water removed therefrom. Likewise, the terms “dried glycol” and “dried monohydric alcohol” respectively refer to a glycol or a monohydric alcohol that has had at least a portion of the water removed therefrom. It is to be recognized that a dried alcoholic component need not necessarily be completely anhydrous when dried, simply that its water content be reduced (e.g., less than 50 wt. % water). In some embodiments, the dried alcoholic component may comprise about 40 wt. % or less water. In some or other embodiments, the dried alcoholic component may comprise about 35 wt. % or less water, or about 30 wt. % or less water, or about 25 wt. % or less water, or about 20 wt. % or less water, or about 15 wt. % or less water, or about 10 wt. % or less water, or about 5 wt. % or less water. In some embodiments of the methods described herein, a substantially anhydrous alcoholic component may be produced upon drying. As used herein, a substance will be considered to be substantially anhydrous if it contains about 5 wt. % water or less.

In other embodiments, it may be more desirable to feed monohydric alcohols to the condensation catalyst due to a lower incidence of coking. As previously described, monohydric alcohols may be more difficult to produce in dried form due to azeotrope formation during distillation. In some embodiments, monohydric alcohols produced from cellulosic biomass solids may be fed directly to a condensation catalyst, without drying. In other embodiments, dried monohydric alcohols may be fed to a condensation catalyst. In some embodiments, dried monohydric alcohols may be produced from dried glycols. Specifically, dried glycols may be produced as described hereinabove, and the dried glycols may then be subjected to a catalytic reduction reaction to produce monohydric alcohols. The monohydric alcohols may contain a comparable amount of water to that present in the dried glycols from which they were formed. Thus, forming dried monohydric alcohols in the foregoing manner may desirably allow a reduced incidence of coking to be realized while maintaining lifetime of the condensation catalyst by providing a dried feed. The foregoing approach for producing dried monohydric alcohols from cellulosic biomass solids is described in commonly owned U.S. Patent Application 61/720,714, filed Oct. 31, 2012 and incorporated herein by reference in its entirety.

In some embodiments, a phenolics liquid phase formed from the cellulosic biomass solids may be further processed. Processing of the phenolics liquid phase may facilitate the catalytic reduction reaction being performed to stabilize soluble carbohydrates. In addition, further processing of the phenolics liquid phase may be coupled with the production of dried glycols or dried monohydric alcohols for feeding to a condensation catalyst. Moreover, further processing of the phenolics liquid phase may produce methanol and phenolic compounds from degradation of the lignin present in the cellulosic biomass solids, thereby increasing the overall weight percentage of the cellulosic biomass solids that may be transformed into useful materials. Finally, further processing of the phenolics liquid phase may improve the lifetime of the slurry catalyst.

Various techniques for processing a phenolics liquid phase produced from cellulosic biomass solids are described in commonly owned U.S. Patent Applications 61/720,689, 61/720,747, and 61/720,774, each filed on Oct. 31, 2012 and incorporated herein by reference in its entirety. As described therein, in some embodiments, the viscosity of the phenolics liquid phase may be reduced in order to facilitate conveyance or handling of the phenolics liquid phase. As further described therein, deviscosification of the phenolics liquid phase may take place by chemically hydrolyzing the lignin and/or heating the phenolics liquid phase in the presence of molecular hydrogen (i.e., hydrotreating) to depolymerize at least a portion of the lignin present therein in the presence of accumulated slurry catalyst. Deviscosification of the phenolics liquid phase may take place before or after separation of the phenolics liquid phase from one or more of the other liquid phases present, and thermal deviscosification may be coupled to the reaction or series of reactions used to produce the alcoholic component from the cellulosic biomass solids. Moreover, after deviscosification of the phenolics liquid phase, the slurry catalyst may be removed therefrom. The catalyst may then be regenerated, returned to the cellulosic biomass solids, or any combination thereof.

In some embodiments, heating of the cellulosic biomass solids and the liquid phase digestion medium to form soluble carbohydrates and a phenolics liquid phase may take place while the cellulosic biomass solids are in a pressurized state. As used herein, the term “pressurized state” refers to a pressure that is greater than atmospheric pressure (1 bar). Heating a liquid phase digestion medium in a pressurized state may allow the normal boiling point of the digestion solvent to be exceeded, thereby allowing the rate of hydrothermal digestion to be increased relative to lower temperature digestion processes. In some embodiments, heating the cellulosic biomass solids and the liquid phase digestion medium may take place at a pressure of at least about 30 bar. In some embodiments, heating the cellulosic biomass solids and the liquid phase digestion medium may take place at a pressure of at least about 60 bar, or at a pressure of at least about 90 bar. In some embodiments, heating the cellulosic biomass solids and the liquid phase digestion medium may take place at a pressure ranging between about 30 bar and about 430 bar. In some embodiments, heating the cellulosic biomass solids and the liquid phase digestion medium may take place at a pressure ranging between about 50 bar and about 330 bar, or at a pressure ranging between about 70 bar and about 130 bar, or at a pressure ranging between about 30 bar and about 130 bar.

### EXAMPLES

To facilitate a better understanding of the present invention, the following examples of preferred embodiments are given. In no way should the following examples be read to limit, or to define, the scope of the invention.

### Example 1

### Bubble Column with Cellulosic Flocculant

An 8-inch diameter×8-foot tall acrylic glass vessel was filled 75% full with deionized water. Cellulosic swimming pool flocculant (nominal 200 mesh) was added at 1%, 2%, 3%, 4% and 5% by weight. Air was sparged at the bottom of the column at 1600 ml/min flowrate, via a central distributor giving nominal 3-mm (⅛ inch bubbles). Video taken at the top of the column showed progressive increase in bubble size as the concentration of flock was increased. By 5 wt % cellulosic flow, bubbles observed breaking through at the top surface were 2.5-3 inches in diameter. Viscosity of the flocculant suspension was measured as approximately 1000 centipoise.

### Example 2

### Nutter Rings in Bubble Column

Example 1 was repeated with addition of 2-feet of 0.7 (inch) Nutter rings as random packing, midway in the column into the settled zone of 5 wt % cellulosic flocculant. Resumption of gas sparging gave much smaller bubbles breaking through to the liquid surface, with diameters less than about 0.75 inch. Shearing of gas bubbles that had coalesced underneath the packed section was evident upon entry to the packed ring section. This example demonstrates the ability of a random packing to shear and break up gas bubbles to a characteristic dimension approximately equal to the packing diameter, or smaller.

### Example 3

### Nutter Rings with Wood Chips

The column was emptied, and refilled with a 1-foot bed of 0.7 (inch) Nutter rings, retained by a 4-mesh screen. Water was added to fill the column and air was sparged beneath the rings at varying rates from 300 to 1200 ml/min using 4 sintered metal spargers (10 micron) distributed across the cross section. Southern pine wood chips were milled via a Retsch cutting mill fitted with 6-mm screen, to a typical dimension of 3-mm by 3-mm by 6 mm. The wood was pre-steamed to a moisture content of 52 wt %.

For example 3A, water was passed downflow through the column at a flowrate of 0.8 ft/min, with a gas sparge rate of 1200 ml/min Wood was added at the top of the column, and allowed to drop onto the zone of Nutter rings. Within 10 minutes of addition, the milled wood had penetrated the ring zone to collect on the 4-mesh retaining screen. A gas pocket developed underneath the retention screen, but continued gas flow re-sheared the gas into bubbles which travelled upward through the ring zone, which was packed with wood. The gas pocket could be released by momentarily stopping the downward flow of liquid, resulting in a release of gas bubbles as the gas was sheared by the Nutter rings. Overall gas bubble dimension was on the order of 10-15 mm.

For example 3B, the liquid flow was reversed to the upflow direction. In this mode, the time required for wood chips to pack the ring section above the retention screen was about 30 minutes. There was no flooding of the bed or collection of gas pockets during this concurrent upflow operation of the bed.

For example 3C, the downflow test was repeated without gas sparging. The rate of transport of wood needles into the ring zone was much slower in the absence of gas sparging, as motive force for rocking the wood particles to allow flow into the ring matrix was now lacking.

This example demonstrates the ability of ring packing to shear gas bubbles to a size less than or equal to the characteristic ring dimension, despite formation of a continuous gas pocket underneath the packed section. This shearing and upward transmission of gas bubbles occurred despite the presence of wood particles within the rings. The presence of wood particles led to a mean gas bubble size that was smaller than ring dimension itself. Downflowing liquid led to some tendency for flooding of the column (retention of gas pockets), which could be immediately reversed by interrupting the downflow of liquid. Gas sparging was observed to assist in the downward migration of wood particles into the ring zone, providing a rocking action to allow the needle shaped particles to dive through the intertwined random packing matrix.

### Example 4

### Nutter Rings with ¼-Inch Wood Chips

Example 3 was repeated with wood minichips produced by chipping directly from debarked Southern pine logs to an average dimension of ¼-inch×½-inch× ⅛ inch. The squarish chips penetrated the rings more slowly than the milled wood “needles,” but penetration was again improved in the presence of gas sparging. Unlike the wood needles, a coalesced gas pocket did not form, given more loose packing present with the squarish minichips.

### Example 5

### Nutter Rings with 10-Mesh Screened Milled Wood

Example 4 was repeated with 0.7-inch Nutter rings and milled wood screened via 10-mm screen. Penetration was slower for the larger particle wood. Wood was less densely packed in the ring zone, with more numerous voids. Gas bubbles were still sheared to a dimension less than or equal to the ring characteristic dimension.

### Example 6

### I-Rings with 10-Mesh Screened Milled Wood

Example 5 was repeated with stainless steel 40-mm I-rings as random packing. With the larger size rings, downward migration of the screened wood particles was more rapid and density of packing as wood migrated to the 4-mesh retention screen was increased, with fewer voids. Gas was again sheared to bubbles less than or equal to the dimension of the ring packing.

### Example 7

### Partially Digested Wood Particles

460-grams of 6-mm screened milled wood (10% moisture) were charged to a 2-gallon Parr reactor, together with 255 grams of methoxypropylphenol, and 3150 grams of deionized water, and heated for 1 hour at 160° C. followed by 1 hour at 190° C., to partially digest the wood. After partial digestion, the remaining softened wood was recovered by filtration, and washed with acetone to remove color bodies and tars.

The washed wood was transferred to a 2-inch diameter glass column, with middle section packed with 15-mm I-rings above a 4-mesh retaining screen. The column was filled with deionized water and air was sparged upflow at 100 ml/min, enabling the partially digested wood to penetrate into the ring zone. Dispersed gas bubbles again penetrated through the 1-foot section of ring packing, despite the presence of deformable, partially digested wood particles in the ring zone.

Examples 1-7 show that use of ring packing enables the shearing of an added gas phase, to a bubble size of the dimension of the ring packing or smaller, in the presence of biomass particles ranging from a uniformly thickened finely divided polymer, to partially digested wood fragments, to rigid wood needles or squarish particles. The biomass particles can penetrate the ring matrix, provided largest dimension of the rings allows the biomass particles to enter the rings. Particle vs ring size can be varied to control the rate of penetration of particles into the ring zone. Liquid downflow can assist in the rate of penetration, but may induce flooding of the gas phase, requiring periodic stoppage of liquid flow to release the trapped gas phase, which is broken up by the ring packing into small bubbles of characteristic dimension equal to or smaller than the ring dimension. Upflow gas sparging assists the downflow migration of woody biomass, via rocking and agitating the dense wood particles to allow their movement downward through the tortuous ring structure.

### Example 8

### Continuous Digester without Packing

A 10-inch diameter×10-foot tall pressure vessel fitted with a 2-mesh screen a foot above the bottom flange was filled with a solvent mixture of 25% tetrahydrofurfural alcohol in deionized water, along with 310 grams of Raney Cobalt 2724 catalyst (WR Grace), and KOH buffer sufficient to maintain a pH between 5 and 6. The reactor was pressured to 1000 psi of H₂, and catalyst-containing liquid was recirculated at 2.5 gallons per minute flowrate. H₂ gas was sparged at the bottom at 30 standard liters per minute flowrate via a sparge ring drilled with 1/16 inch holes. Excess hydrogen was vented from the top of the reactor. The reactor was heated to 225° C. via an electric heater on the recirculation loop. To initiate reaction, Southern pine wood chips (nominal 55% moisture) of nominal 1 inch×1.5 inch×⅛ inch size were added at a rate of 1 lb/hr for the first day, followed by a 2 lb/hr feed rate.

Excess inventory was removed on level control via 10-micron crossflow filter, to retain catalyst. After 8 days of operation, a sample of crossflow filter product was analyzed by gas chromatography, using a 60-m×0.32 mm ID DB-5 column of 1 μm thickness, with 50:1 split ratio, 2 ml/min helium flow, and column oven at 40° C. for 8 minutes, followed by ramp to 285° C. at 10° C./min, and a hold time of 53.5 minutes. The injector temperature was set at 250° C., and the detector temperature was set at 300° C. Gas Chromatographic—Mass Spec (GCMS) was effected using the same protocol. Results indicated negligible formation of expected ethylene glycol and 1,2-propylene glycol products, expected to form from the hydrocatalytic conversion of carbohydrates hemicellulose and cellulose present in the wood feed.

A sample was distilled using a 1-liter 3-necked flash fitted with short path Vigreux column, first at atmospheric pressure under a small blanket of nitrogen, then under vacuum with increase in bottoms temperature to 350° C. A substantial heavy residue was present, representing more than 60% of the wood and derivatives present in the reactor (dry basis).

### Example 9

### Continuous Digester with Nutter Rings

Example 8 was repeated, but with a 7-inch zone of 0.7-inch Nutter rings retained 1 foot off the bottom of the reactor, and another 5-inch zone retained 2 feet above the bottom of the reactor. In addition, the system included a relatively short internal draft tube, similar to FIG. 1, extending through the bottom ring bed, a stinger between the ring beds, and an external fluid conduit for catalyst recirculation lines. GC analysis after 8 days of operation revealed the presence of ethylene glycol, 1,2-proplene glycol, light C₁-C₃ monooxygenates, intermediate C₄-C₆ monooxygenates (ketones, alcohols) and diols, and formation of some methoxypropylphenol. Observed liquid phase product formation accounted for approximately 67% of the carbon present in the carbohydrate portion of the wood feed.

A distillation sample revealed the presence of 31% residue relative to the expected concentration based on the dry weigh of wood feed. Since the wood sample is approximately 30% lignin, which is only minimally converted in the process to components capable of being distilled overhead, the bottoms residue from batch distillation of crossflow product contained minimal heavy ends/tar above the expected lignin polymer.

Example 9 shows the value of reactor packing in providing effective contacting and mass transfer of hydrogen gas, in order to selectively hydrogenate intermediates derived from the hydrothermal digestion of biomass and to obtain intermediates capable of being distilled overhead to separate from heavy ends and ash.

### Example 10

### Acid Condensation of Biomass Reaction Intermediates to Liquid Biofuels

Intermediate products from the hydrothermal digestion and reaction of southern pine wood in the presence of Raney Cobalt catalyst and hydrogen were vaporized and passed over a bed of amorphous silica alumina catalyst, followed by ZSM-5, at WHSV of 0.5, at a nominal pressure of 75 psi, and temperature of 325-375° C., as disclosed in co-pending application Ser. No. 62/186,919, filed on 30 Jun. 2015.

This example showed production of an aromatic-rich liquid biofuel of components in the gasoline and diesel range, from acid condensation of components formed via biomass digestion in the presence of ring packing.

As can be seen, present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods may also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

We claim:

1. A method comprising:
a) introducing cellulosic biomass solids to a hydrothermal digestion unit comprising:
i) a reactor;
ii) a gas feed line for providing gas to the reactor;
iii) a biomass feed system for feeding biomass into the reactor;
iv) a fluid circulation system including a fluid inlet, a pump, and at least one fluid injector, wherein at least said fluid inlet and said fluid injector are in fluid communication with said pump and are within said reactor; and
v) a screen positioned within the reactor and defining a lower zone therebelow;
b) providing a liquid phase digestion medium containing a slurry catalyst in the hydrothermal digestion unit, the slurry catalyst being capable of activating molecular hydrogen;
c) providing a layer of organic solvent in the hydrothermal digestion unit, said organic solvent being less dense than and substantially immiscible with said digestion medium, whereby said organic solvent forms an organic layer floating on said digestion medium;
d) circulating said liquid phase digestion medium through said fluid circulation system;
e) supplying an upwardly directed flow of molecular hydrogen through the cellulosic biomass solids; and
f) maintaining the cellulosic biomass solids and slurry catalyst at a temperature sufficient to cause digestion of cellulosic biomass solids into an alcoholic component;
g) operating the hydrothermal digestion unit such that the level of the organic layer in said reactor is below the top of the reactor, thereby leaving a gas-filled headspace;
h) wherein the hydrothermal digestion unit further comprises:
1) at least one first eductor having a first inlet in the headspace and an outlet below the first inlet;
2) at least one second eductor having a second inlet in the organic layer and an outlet below said second inlet end;
3) at least one downdraft tube having a third inlet in the digestion medium and an outlet in the lower zone;
wherein fluid flowing from said fluid injector entrains gas from said headspace into said first eductor;
wherein fluid flowing out of said first eductor draws fluid from said organic layer into said second eductor;
wherein fluid flowing out of said second eductor draws fluid from said digestion medium into said downdraft tube, and
fluid flowing out of said downdraft tube is discharged into said reactor below said packing material.

2. The method according to claim 1, further including the step of operating the system such that the flow rate of fluids in said draft tube is sufficient to carry the entrained gas to said lower zone.

3. The method according to claim 1 wherein said fluid circulation system removes slurry from said lower zone via said fluid inlet and transmits the removed slurry to a separator that separates the slurry into a catalyst concentrate stream and a stream that is relatively low in solids, wherein said catalyst concentrate stream is fed back into said reactor at a point that is above the packed bed, and wherein the low-solids stream is injected into the reactor via said first fluid injector.

4. The method according to claim 1 wherein at least a portion of either said fluid circulation system or said downdraft tube is outside of said reactor.

5. The method according to claim 2 wherein from one-half up to all of the volume of slurry removed from said lower zone is returned to reactor via said first fluid injector.

6. The method according to claim 1, further including the step of removing a portion of said organic layer from said reactor, removing lignin therefrom so as to generate a lignin-reduced organic steam, and returning at least a portion of said lignin-reduced organic stream to said reactor.

7. A method comprising:
a) introducing cellulosic biomass solids to a hydrothermal digestion unit comprising:
i) a reactor;
ii) a gas feed line for providing gas to the reactor;
iii) a biomass feed system for feeding biomass into the reactor;
iv) a fluid circulation system including a fluid inlet, a pump, and at least one fluid injector, wherein at least said fluid inlet and said fluid injector are in fluid communication with said pump and are within said reactor;
v) a screen positioned within the reactor defining a lower zone therebelow; and
vi) a bed of reactor packing material resting on said screen;
b) providing a liquid phase digestion medium containing a slurry catalyst in the hydrothermal digestion unit, the slurry catalyst being capable of activating molecular hydrogen;
c) providing a layer of organic solvent in the hydrothermal digestion unit, said organic solvent being less dense than and substantially immiscible with said digestion medium, whereby said organic solvent forms an organic layer floating on said digestion medium;
d) circulating said liquid phase digestion medium through said fluid circulation system;
e) supplying an upwardly directed flow of hydrogen gas through the cellulosic biomass solids;
f) maintaining the cellulosic biomass solids and slurry catalyst at a temperature sufficient to cause digestion of cellulosic biomass solids into an alcoholic component;
g) operating the hydrothermal digestion unit such that a liquid-liquid extraction is accomplished through contact between the solvent phase and the digestion medium, a bubble column effect is achieved as a result of gas upward through said digestion medium and said packed bed serves to enhance contact between the biomass and the gas.
